(19) United States
(12) Patent Application Publication
(10) Pub. No.: US 2026/0266830 A1
(43) Pub. Date: Sep. 10, 2026

(54) COMPOSITIONS OF BINDING REAGENTS AND METHODS OF USE

(71) Applicant: Nautilus Subsidiary, Inc., Seattle, WA (US)

(72) Inventors: Julia K. ROBINSON, Portola Valley, CA (US); Rajesh KOTA, San Diego, CA (US); Samuel Hyde GRILLO, Redwood City, CA (US); Maureen NEWMAN, Belmont, CA (US); Jonathan B. LEANO, Foster City, CA (US); Zhengjian ZHANG, Albany, CA (US); Scott Michael TABAKMAN, San Diego, CA (US); Fletcher BAIN, Redwood City, CA (US); Filip BARTNICKI, Redwood City, CA (US); Kevin MCVEY, Seattle, WA (US); Jacinto VILLANUEVA, Pleasant Hill, CA (US); Ilaria SANDEI, Redwood City, CA (US); Keith BJORNSON, Fremont, CA (US); Steven TAN, San Mateo, CA (US); Seok Ki CHOI, Palo Alto, CA (US); Terren R. CHANG, Redwood City, CA (US)

(21) Appl. No.: 19/560,958

(22) Filed: Mar. 9, 2026

Related U.S. Application Data
(60) Provisional application No. 63/769,587, filed on Mar. 10, 2025.

(51) Int. Cl. G01N 33/58; G01N 33/543

(52) U.S. Cl. CPC G01N 33/582; G01N 33/54386

(57) ABSTRACT

Methods, compositions, and systems containing detectable probes are provided. The detectable probes may include one or more affinity reagents joined to one or more detectable labels, optionally by a linking moiety. The detectable probes provided herein may be useful for various analyte characterization assays, including certain single molecule detection assays.

### CROSS-REFERENCE TO RELATED APPLICATIONS

This application claims priority to U.S. Provisional Application No. 63/769,587, filed on Mar. 10, 2025, which is incorporated herein by reference in its entirety.

### BACKGROUND

Binding reagents can encompass a class of molecules that may be useful to certain assays due to their forming binding interactions with binding partners. A binding reagent may be as simple as an unmodified affinity reagent, such as an antibody or aptamer. However, binding reagents can be formulated with increasing complexity. For example, multiple affinity reagents (e.g., antibodies, aptamers, etc.) may be joined into a single complex via attachment to a linking particle or molecule. Further, a binding reagent may be made detectable by attaching an affinity reagent to a detectable label, such as a fluorophore, luminophore, or a barcode moiety (e.g., a nucleic acid barcode or a peptide barcode).

Useful compositions of binding reagents for certain assays are described in U.S. Pat. No. 10,473,654 and 11,692,217, an U.S. Patent Publication Nos. 20230090454A1, 20240053333A1, 20240280568A1, 20240426839A1, and 20250066841A1, each of which is herein incorporated by reference in its entirety.

### SUMMARY

In an aspect, provided herein is a method of forming a detectable probe, comprising: (a) providing an antibody having a binding specificity for an epitope of 2, 3, 4, or 5 amino acids in length, (b) attaching a detectable label to the antibody in a site-directed manner, wherein the detectable label comprises a polymer strand and one or more luminophores attached to the polymer strand.

In another aspect, provided herein is a method of forming a detectable probe, comprising: (a) providing an antibody having a binding specificity for two or more differing proteins of a proteome, wherein the two or more differing proteins of the proteome are not differing isoforms or differing proteoforms of a protein, (b) attaching a detectable label to the antibody in a site-directed manner, wherein the detectable label comprises a polymer strand and one or more luminophores attached to the polymer strand.

In another aspect, provided herein is a method of characterizing a plurality of analytes, comprising: (a) providing a solid support comprising the plurality of analytes, wherein the plurality of analytes is immobilized on the solid support, and wherein each analyte of the plurality of analytes is immobilized at a unique address of the solid support such that the analyte is detectable at single-analyte resolution, (b) contacting a sequence of detectable probes to the solid support, wherein the sequence of detectable probes comprises two or more detectable probes that differ with respect to binding specificity, wherein each detectable probe of the sequence of detectable probes comprises an affinity reagent attached to a detectable label in a site-specific manner, and wherein the detectable label of each detectable probe comprises a polymer strand attached to one or more luminophores, (c) at each address of the solid support containing an immobilized analyte of the plurality of analytes, detecting a presence or absence of each detectable probe of the sequence of detectable probes, (d) based upon the detected presence or absence of binding for each detectable probe of the sequence of detectable probes to each analyte of the plurality of analytes, characterizing each analyte of the plurality of analytes.

In another aspect, provided herein is a system for analyte characterization, comprising: (a) a solid support comprising the plurality of analytes, wherein the plurality of analytes is immobilized on the solid support, and wherein each analyte of the plurality of analytes is immobilized at a unique address of the solid support such that the analyte is detectable at single-analyte resolution, (b) one or more vessels, each vessel of the one or more vessels comprising a detectable probe composition, wherein each detectable probe of the detectable probe composition comprises an affinity reagent attached to a detectable label in a site-specific manner, and wherein the detectable label of each detectable probe comprises a polymer strand attached to one or more luminophores, (c) a fluidic system, wherein the fluidics system provides fluidic communication between the solid support and the one or more vessels, (d) an optical detection device, and (e) a processor in communication with the optical detection device, wherein the processor is configured to received signal information from the optical detection device and determine the presence or absence of a detectable probe of the detectable probe composition at each unique address of the solid support.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A-1B illustrate various configurations of multivalent affinity reagents and binding reagents containing multivalent affinity reagents, in accordance with some embodiments.

FIGS. 2A-2B depict a method of forming a linking moiety for a binding reagent comprising a plurality of detectable labels, in accordance with some embodiments.

FIGS. 3A, 3B, 3C, 3D, and 3E display configurations of clustered label compositions, in accordance with some embodiments.

FIG. 4 shows a method of attaching detectable labels to affinity reagents in a semi-stochastic manner, in accordance with some embodiments.

FIGS. 5A-5B illustrate configurations of a system that utilizes a quenching agent to inhibit autofluorescence, in accordance with some embodiments.

FIGS. 6A-6B depict configurations of binding reagents containing components that are attached by a chelating chemistry, in accordance with some embodiments.

FIGS. 7A, 7B, 7C, 7D, 7E, 7F, 7G, 7H, and 7I display steps of methods of dissociating a binding reagent from a binding target utilizing a photolabile moiety, in accordance with some embodiments.

FIG. 8 shows a configuration of a binding reagent, in accordance with some embodiments.

FIGS. 9A-9B illustrate configurations of binding reagents containing clustered label compositions, in accordance with some embodiments.

FIGS. 10A, 10B, 10C and 10D depict configurations of binding reagents containing clustered label compositions, in accordance with some embodiments.

FIG. 11 displays a configuration of a binding reagent, in accordance with some embodiments.

FIG. 12 shows a configuration of a binding reagent utilizing a fluorescently-labeled coupling moiety, in accordance with some embodiments.

FIGS. 13A-13B illustrate configuration of binding reagents utilizing phiton detectable label compositions, in accordance with some embodiments.

FIG. 14 depicts a configuration of a binding reagent containing a fluorescent nanoparticle, in accordance with some embodiments.

FIG. 15 displays a configuration of a binding reagent, in accordance with some embodiments.

FIGS. 16A, 16B, and 16C show various configurations of clustered moiety composition, in accordance with some embodiments.

FIG. 17 illustrates a system that can utilize a binding reagent composition set forth herein, in accordance with some embodiments.

FIG. 18 depicts a reaction scheme for synthesis of a PEGylated cluster dye, in accordance with some embodiments.

FIG. 19 displays signal intensity histograms for differing probe structures based upon the style of attachment of detectable labels to the probes, in accordance with some embodiments.

### DETAILED DESCRIPTION

The present disclosure provides compositions of binding reagents, methods of formation of the provided binding reagents, methods of use of the provided binding reagents, and systems that contain the provided binding reagents. The provided binding reagents may be useful for molecular characterization, such as methods of characterizing populations of biomolecules (e.g., nucleic acids, polypeptides, glycopeptides, polysaccharides, etc.). Also provided herein is a method, comprising: a) contacting a binding reagent composition set forth herein to a plurality of analytes, b) binding the binding reagent composition to an analyte of the plurality of analytes, and c) detecting the binding of the composition to the analyte of the plurality of analytes.

Identifying binding reagents that are suitable for certain assays is a common challenge. Depending upon the assay configuration, optimal binding reagent configuration may be a balance between certain physical properties of the binding reagent, including binding specificity, binding kinetics, mass transfer characteristics (e.g., diffusion coefficient), and detection characteristics (e.g., luminescence magnitude, quantum efficiency, etc.). In some cases, optimizing for a particular physical property of a binding reagent may deoptimize another physical property. For example, attaching a quantity of fluorophores to an affinity reagent to achieve a sufficient fluorescence magnitude for optical detection will increase the molecular weight and likely the occupied volume of a binding reagent containing the affinity reagent, thereby slowing the diffusion of the binding reagent. Further, it may be necessary to decorate the fluorophores onto a molecular scaffold to inhibit fluorescence self-quenching, further increasing the molecular weight and/or occupied volume of the binding reagent. In another example, an affinity reagent may be directly labeled by attaching fluorophores or luminophores to the affinity reagent structure. However, if a fluorophore or luminophore becomes attached too close to the ligand-binding portion of the affinity reagent, the binding kinetics of the affinity reagent may be altered, or binding may be altogether inhibited by the presence of the detectable label.

Furthermore, the manner in which detectable labels are attached to a binding reagent can affect the behavior of the binding reagent. Stochastic labeling methods can cause labeling of residues that give rise to the binding characteristic of a binding reagent, thereby disabling the binding reagent. The effective concentration of a binding reagent may be lower than the absolute concentration of the binding reagent due to the presence of the disabled binding reagents. Site-directed methods of labeling binding reagents, such as attachment of labels to terminal moieties or other portions of the binding reagent that do not participate in binding, may be preferable to reduce the amount of loss of effective binding reagents.

Optimal characteristics of a binding reagent may further depend upon the type of assay in which the binding reagent is utilized. Optimal characteristics for a bulk-resolution assay may differ from those for a single-molecule resolution assay. Moreover, differing detection techniques (e.g., fluorescence microscopy, total internal reflectance, surface plasmon resonance, biolayer interferometry, etc.) may have differing criteria for a suitable binding reagent given differences in detection modes (e.g., sampling rate, spatial resolution, etc.).

Two differing affinity reagents may have substantially different behaviors when formulated into a detectable probe in the same manner. Non-specific or off-target binding behaviors of two differing affinity reagents can vary substantially. Accordingly, the optimal formulation for any given detectable probe containing an affinity reagent can vary as well. The present disclosure provides numerous configurations of detectable probes. For any given affinity reagent, the skilled person can readily test for the on-target, off-target, and non-specific binding behaviors of differing probe formulations to identify the most preferable formulation for a given assay.

The present disclosure describes configurations of binding reagents that may be useful for certain assays. Various configurations for attaching detectable labels to binding reagents are provided. Also provided are various configurations of linking moieties that are suitable for joining binding reagent components, including one or more affinity reagents, one or more detectable labels, and one or more tether strands. The present disclosure also provides methods of forming the provided binding reagents. The present disclosure further provides methods of utilizing the binding reagents, and systems that are configured to implement methods of utilizing binding reagents set forth herein. The present disclosure further provides a kit comprising one or more binding reagent compositions, as set forth herein.

Terms used herein will be understood to take on their ordinary meaning in the relevant art unless specified otherwise. Several terms used herein and their meanings are set forth below.

The term “comprising” is intended herein to be open-ended, including not only the recited elements, but further encompassing any additional elements.

In some embodiments set forth herein, the term “each,” when used in reference to a collection of items, is intended to identify an individual item in the collection but does not necessarily refer to every item in the collection. Exceptions can occur if explicit disclosure or context clearly dictates otherwise.

In some embodiments set forth herein, the terms “site” or “address” can refer to a location in an array where a particular analyte (e.g., protein, peptide or unique identifier label) is present. An address can contain a single analyte, or it can contain a population of several analytes of the same species (i.e., an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

In some embodiments set forth herein, the term “affinity reagent” can refer to a molecule or other substance that is capable of specifically or reproducibly binding to an analyte (e.g., protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

In some embodiments set forth herein, the term “array” can refer to a population of analytes (e.g., proteins) that are associated with unique identifiers such that the analytes can be distinguished from each other. A unique identifier can be, for example, a solid support (e.g., particle or bead), address on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent bonds or non-covalent bonds (e.g., ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

In some embodiments set forth herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some embodiments set forth herein, the term “binding affinity” or “affinity” can refer to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

In some embodiments set forth herein, the term “binding probability” can refer to the probability that an affinity reagent or probe may be observed to interact with an analyte, for example, within a given binding context. A binding probability may be expressed as a discrete number (e.g., 0.4 or 40%), a matrix of discrete numbers, or as a mathematical model (e.g., a theoretical or empirical model). A binding probability may include one or more factors, including binding specificity, likelihood of locating a target epitope, or the likelihood of binding for a sufficient time to detect a binding interaction.

In some embodiments set forth herein, the term “binding profile” can refer to a plurality of binding outcomes for a protein or other analyte. The binding outcomes can be obtained from independent binding observations, for example, independent binding outcomes can be acquired using different affinity reagents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

In some embodiments set forth herein, the term “binding specificity” can refer to the tendency of a binding reagent to preferentially interact with a given analyte relative to other analytes. A binding reagent may have a calculated, observed, known, or predicted binding specificity for a given analyte. Binding specificity may refer to selectivity for a single analyte in a given sample relative to one, some or all other analytes in the sample. Moreover, binding specificity may refer to selectivity for a subset of analytes in a given sample relative to at least one other analyte in the sample.

In some embodiments set forth herein, the term “click reaction” can refer to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −100 kJ/mol, −250 kJ/mol, −500 kJ/mol, or less. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in U.S. Patent Publication No. 2021/0101930 A1, which is incorporated herein by reference.

In some embodiments set forth herein, the term “epitope” can refer to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, miniprotein or other affinity reagent. An epitope can optionally bind an antibody to elicit an immune response. However, an epitope need not necessarily participate in, nor be capable of, eliciting an immune response.

In some embodiments set forth herein, the term “fluid-phase,” when used in reference to a molecule, means the molecule is in a state wherein it is mobile in a fluid, for example, being capable of diffusing through the fluid.

In some embodiments set forth herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

In some embodiments set forth herein, the term “immobilized,” when used in reference to a molecule that is in contact with a fluid phase, can refer to the molecule being prevented from diffusing in the fluid phase. For example, immobilization can occur due to the molecule being confined at, or attached to, a solid phase. Immobilization can be temporary (e.g., for the duration of one or more steps of a method set forth herein) or permanent. Immobilization can be reversible or irreversible under conditions utilized for a method, system or composition set forth herein.

In some embodiments set forth herein, the term “label” can refer to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some embodiments set forth herein, the terms “linking moiety” or “linker” can refer to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

In some embodiments set forth herein, the term “measurement outcome” can refer to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome includes an empirical or putative measurement outcome for a candidate analyte (e.g., for a candidate protein) that is known or suspected of being present in a sample or assay. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

In some embodiments set forth herein, the term “nucleic acid origami” can refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some embodiments set forth herein, the term “nucleic acid tag” can refer to a nucleic acid molecule or sequence that is encoded with information that uniquely identifies an object with which it is associated. A nucleic acid tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The nucleic acid tag can be, for example, DNA, RNA or analogs thereof. The length of the tag sequence can be at least about 5, 8, 10, 15, 20, 25, 30, 40, 50, 75, 100 or more nucleotides. Alternatively or additionally, the length of the tag sequence can be at most about 100, 75, 50, 40, 30, 25, 20, 15, 10, 8, 5 or fewer nucleotides.

In some embodiments set forth herein, the term “post-translational modification” can refer to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

In some embodiments set forth herein, the term “promiscuous,” when used in reference to a reagent, means that the reagent is known or suspected to react with a variety of different analytes in a given sample. For example, an affinity reagent that is known or suspected to recognize a variety of different analytes (e.g., a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample.

In some embodiments set forth herein, the term “protein” can refer to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

In some embodiments set forth herein, the term “single,” when used in reference to an object such as an analyte, means that the object is individually manipulated or distinguished from other objects. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some embodiments set forth herein, the term “single-analyte resolution” can refer to the detection of, or ability to detect, an analyte on an individual basis, for example, as distinguished from its nearest neighbor in an array.

In some embodiments set forth herein, the terms “site-directed” and “site-specific,” when used in reference to a binding reagent or a plurality thereof, can refer to the attachment of one or more detectable labels to a specific residue or moiety of a binding reagent or affinity reagent. Conversely, site-directed or site-specific modification can refer to not attaching a detectable label to a specific residue or moiety of a binding reagent or affinity reagent. With respect to a population of binding reagents or affinity reagents, site-directed or site-specific modification can refer to each binding reagent of the population of binding reagents or affinity reagents having a detectable label attached to an equivalent residue or moiety. Site-directed or site-specific modification of binding reagents or affinity reagents can differ from stochastic modification with respect to uniformity of modification. A population of stochastically-modified binding reagents or affinity reagents may vary with respect to which residues or moieties are modified and/or what quantity of residues or moieties are modified while a population of site-directed or site-specifically modified binding reagents or affinity reagents will consistently be modified at the same residue or moiety or a set thereof.

In some embodiments set forth herein, the term “solid support” can refer to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides, etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

In some embodiments set forth herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some embodiments set forth herein, the term “type,” when used in reference to a subset of analytes, can refer to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

In some embodiments set forth herein, the term “unique identifier” can refer to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g., ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

In some embodiments set forth herein, the term “unique identifier label” can refer to a unique identifier that is a particle, molecule or moiety that provides a detectable characteristic. The detectable characteristic can be, for example, an optical signal such as absorbance of radiation, luminescence (e.g., fluorescence) emission, luminescence lifetime, luminescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

In an aspect, provided herein is a method of forming a detectable probe, comprising: (a) providing an antibody having a binding specificity for an epitope of 2, 3, 4, or 5 amino acids in length, (b) attaching a detectable label to the antibody in a site-directed manner, wherein the detectable label comprises a polymer strand and one or more luminophores attached to the polymer strand.

In another aspect, provided herein is a method of forming a detectable probe, comprising: (a) providing an antibody having a binding specificity for two or more differing proteins of a proteome, wherein the two or more differing proteins of the proteome are not differing isoforms or differing proteoforms of a protein, (b) attaching a detectable label to the antibody in a site-directed manner, wherein the detectable label comprises a polymer strand and one or more luminophores attached to the polymer strand.

In another aspect, provided herein is a method of characterizing a plurality of analytes, comprising: (a) providing a solid support comprising the plurality of analytes, wherein the plurality of analytes is immobilized on the solid support, and wherein each analyte of the plurality of analytes is immobilized at a unique address of the solid support such that the analyte is detectable at single-analyte resolution, (b) contacting a sequence of detectable probes to the solid support, wherein the sequence of detectable probes comprises two or more detectable probes that differ with respect to binding specificity, wherein each detectable probe of the sequence of detectable probes comprises an affinity reagent attached to a detectable label in a site-specific manner, and wherein the detectable label of each detectable probe comprises a polymer strand attached to one or more luminophores, (c) at each address of the solid support containing an immobilized analyte of the plurality of analytes, detecting a presence or absence of each detectable probe of the sequence of detectable probes, (d) based upon the detected presence or absence of binding for each detectable probe of the sequence of detectable probes to each analyte of the plurality of analytes, characterizing each analyte of the plurality of analytes.

In another aspect, provided herein is a system for analyte characterization, comprising: (a) a solid support comprising the plurality of analytes, wherein the plurality of analytes is immobilized on the solid support, and wherein each analyte of the plurality of analytes is immobilized at a unique address of the solid support such that the analyte is detectable at single-analyte resolution, (b) one or more vessels, each vessel of the one or more vessels comprising a detectable probe composition, wherein each detectable probe of the detectable probe composition comprises an affinity reagent attached to a detectable label in a site-specific manner, and wherein the detectable label of each detectable probe comprises a polymer strand attached to one or more luminophores, (c) a fluidic system, wherein the fluidics system provides fluidic communication between the solid support and the one or more vessels, (d) an optical detection device, and (e) a processor in communication with the optical detection device, wherein the processor is configured to received signal information from the optical detection device and determine the presence or absence of a detectable probe of the detectable probe composition at each unique address of the solid support.

A binding reagent composition set forth herein may provide improved performance in certain assays including, in particular, single-molecule assays. In some cases, the performance of a detectable probe may be measured by internal standards that contain an epitope bound by the detectable probe. For example, an array, as set forth herein, may be provided with a plurality of standard analytes. Binding of detectable probes to standard analytes may be detected analogously to the detection of detectable probes to analytes of interest. A detectable probe set forth herein may bind available targets above a threshold percentage. The threshold percentage may be at least about 2× (e.g., at least about 5×, 10×, 20×, 50×, 100×, or more than 100×) a non-specific binding rate of the detectable probe. In some cases, a detectable probe may bind at least about 1%, 2%, 5%, 10%, 20%, 25%, 50%, or more than 50% of available targets (e.g., standard analytes containing an epitope bound by the detectable probe).

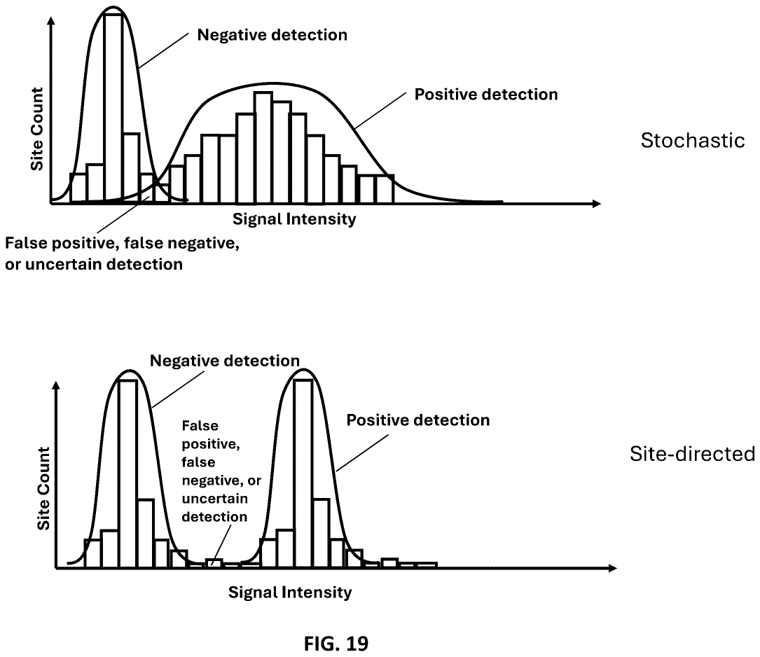

Detectable probe compositions set forth herein be characterized as having improved signal intensity due to reduced fluorophore quenching. Likewise, certain probe compositions, such as those containing site-directed label attachment, may provide a more consistent or less variable signal intensity amongst a population of detectable probes. FIG. 19 provides exemplary signal intensity histograms for stochastically-labeled probes and site-specific labeled probes. Such histograms may be determined by an image processing algorithm implemented on a system set forth herein. An image processing algorithm may quantify the number of discrete analytes or array addresses having a particular detected signal intensity from detection data. The histograms may be divided into separate distributions of negative detections or non-binding events (lower intensity signals) and positive detections or binding events (higher intensity signals). FIG. 19 shows an exemplary method in which Gaussian curves are overlayed on the intensity histograms. The overlapping area between the negative detection Gaussian curve and the positive detection Gaussian curve may represent the approximate quantity or percentage of sites that cannot be confidently assigned as containing a presence or absence of a bound detectable probe. Although FIG. 19 depicts curve-fitting by Gaussian distributions, the method of analysis for quantifying positive or negative binding detections is not particularly limited. The stochastic case can lead to a greater amount of variability in number of detectable labels attached to each detectable probe compared to site-directed methods, so a broader distribution of signal intensities for positive detections may be observed.

Accordingly, a detectable probe composition when utilized in an analyte characterization method, as set forth herein, may produce a clear signal separation when analyzed by an image processing algorithm, as evidenced by the quantity of sites that can be provided a confident binding or non-binding classification (i.e., presence of a detectable probe bound to an analyte; absence of a detectable probe bound to an analyte). A method may comprise one or more steps of: (i) contacting a plurality of detectable probes, as set forth herein, to an array of analytes, as set forth herein, wherein a plurality of analytes is immobilized on the array of analytes, (ii) detecting for each site of the array of analytes a value of an optical signal, (iii) for each site of the array of analytes, classifying the value of the optical signal as a presence of an optical signal, an absence of an optical signal, false positive (e.g., higher intensity signal but no bound detectable probe), false negative (e.g., lower intensity signal but bound detectable probe), or uncertain, wherein at least about 80% (e.g., at least about 85%, 90%, 95%, 98%, 99%, 99.5%, 99.9%, or more than 99.9%) of the sites of the array of analytes have a classification of either presence of the optical signal or absence of the optical signal. Alternatively, a method may comprise a step of: (iii) for each site of the array of analytes, classifying the value of the optical signal as a presence of an optical signal, an absence of an optical signal, false positive (e.g., higher intensity signal but no bound detectable probe), false negative (e.g., lower intensity signal but bound detectable probe), or uncertain, wherein no more than about 20% (e.g., no more than about 15%, 10%, 5%, 1%, 0.5%, 0.1%, or less than 0.1%) of the sites of the array of analytes have a classification of either false positive, false negative, or uncertain.

Certain detectable probe compositions may be particularly useful for detection in well-based detections systems, such as zero-mode waveguides. Such systems typically containing a region near the bottom of the well in which an optical signal is detectable, and a region of the well in which optical signals are inhibited or non-detectable. Detectable probe compositions with small or compact structures, such as those based on dendrimeric or clustered polymers, may provide a greater quantity of dye molecules in the portion of a well from which a fluorescent signal can be detected, whereas a larger probe structure (e.g., a probe containing a nucleic acid origami) may locate some or all dye molecules beyond the detection zone of the well.

The present disclosure provides detectable probes that may be useful for certain analyte characterization methods, including those set forth herein. In some cases, a detectable probe provided herein can have a binding specificity for an epitope of 2, 3, 4, or 5 amino acids in length. In some cases, a detectable probe provided herein can have a binding specificity for two or more differing proteins of a proteome, wherein the two or more differing proteins of the proteome are not differing isoforms or differing proteoforms of a protein. In some cases, a detectable probe provided herein can have a binding specificity for a particular isoform or proteoform of a protein. In some cases, a detectable probe provided herein can have a binding specificity for an epitope comprising a post-translational modification.

A binding reagent may comprise an affinity reagent. An affinity reagent may comprise any suitable binding molecule, such as an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, Major Histocompatibility Complex (MHC), DARPin, monobody, nanoCLAMP, lectin, carbohydrate, SpyCatcher or SpyTag. A binding reagent may be directly attached to one or more of: a linking moiety, a detectable label, and a tether strand.

A binding reagent may comprise only one affinity reagent. A binding reagent may comprise only one affinity reagent, in which the affinity reagent is not directly attached to a linking moiety. A binding reagent may comprise only one affinity reagent, in which the affinity reagent is not directly attached to a detectable label. A binding reagent may comprise only one affinity reagent, in which the affinity reagent is not directly attached to a tether strand.

A binding reagent may comprise two or more affinity reagents. A binding reagent may comprise two or more affinity reagents, in which the two or more affinity reagents are structurally identical. A binding reagent may comprise two or more affinity reagents, in which the two or more affinity reagents have the same binding specificity for an analyte. A binding reagent may comprise two or more affinity reagents, in which the two or more affinity reagents have the same binding specificity for an epitope (e.g., an epitope that is present in multiple differing analytes). Two or more affinity reagents of a binding reagent may be joined to each other by a linking moiety. A binding reagent may comprise two or more affinity reagents and one or more of: i) a detectable label, and ii) a tether strand, in which the detectable label and/or the tether strand is not directly attached to an affinity reagent of the two or more affinity reagents. A binding reagent may comprise two or more affinity reagents and one or more of: i) a detectable label, and ii) a tether strand, in which the detectable label and/or the tether strand is not directly attached to either affinity reagent of the two or more affinity reagents. A binding reagent may comprise two or more affinity reagents, a linking moiety, and one or more of: i) a detectable label, and ii) a tether strand, in which the two or more affinity reagents, the detectable label, and/or the tether strand are attached to the linking moiety.

A binding reagent may comprise at least about 1, 2, 3, 4, 5, 6, 7, 8, 9, 10, 15, 20, 50, 100, or more than 100 affinity reagents. Alternatively or additionally, a binding reagent may comprise no more than about 100, 50, 20, 15, 10, 9, 8, 7, 6, 5, 4, 3, 2, or less than 2 affinity reagents.

In an aspect, provided herein is a composition, comprising: a) a linking moiety, and b) two or more affinity reagents, wherein each affinity reagent of the two or more affinity reagents is attached to the linking moiety, wherein an affinity reagent of the two or more affinity reagents comprises an antibody or a functional fragment thereof, wherein the antibody or the functional fragment thereof comprises: i) a heavy chain, the heavy chain comprising at least two variable fragments, the at least two variable fragments attached by a linker; and ii) at least two light chains, the at least two light chains attached to the heavy chain, wherein each of the at least two variable fragments is attached to a light chain of the at least two light chains, and wherein each light chain attached to a variable fragment forms a paratope.

In some embodiments set forth herein, the term “valency” can refer to the quantity of paratopes present in an affinity reagent or binding reagent. Valency can refer to the quantity of paratopes present in an affinity reagent or binding reagent for a single epitope or ligand of a binding partner. For example, a single nucleic acid aptamer may have a valency of 1 due to having a single ligand-binding nucleotide sequence. In another example, a full antibody can have a valency of at least 2 due to having at least 1 paratope on each arm of the antibody. An affinity reagent can have a valency of at least about 1, 2, 3, 4, 5, 6, 7, 8, 9, 10, 15, 20, 25, 50, 100, or more than 100. Alternatively or additionally, an affinity reagent can have a valency of no more than about 100, 50, 25, 20, 15, 10, 9, 8, 7, 6, 5, 4, 3, 2 or less than 2. A binding reagent may have a total valency (i.e., a sum of the valencies of all affinity reagents present in the binding reagent) of at least about 1, 2, 3, 4, 5, 10, 15, 20, 25, 30, 35, 40, 45, 50, 75, 100, 125, 150, 200, 300, 400, 500, 1000, or more than 1000. Alternatively or additionally, a binding reagent may have a total valency of no more than about 1000, 500, 400, 300, 200, 150, 125, 100, 75, 50, 45, 40, 35, 30, 25, 20, 15, 10, 5, 4, 3, 2, or less than 2.

A binding reagent may comprise an affinity reagent, in which the affinity reagent is engineered to have an increased valency. For example, two or more nucleic acid aptamers may be joined to form a multivalent aptamer. In another example, an antibody may be modified with extended heavy chains that can each bind more than one light chain, thereby providing at least two complementarity-determining regions to an arm of the antibody. If two extended heavy chains are brought together to form a full antibody, the antibody can have a valency of at least 4. Constructs of antibodies with engineered increased valencies are described in U.S. Patent Publication No. US20200157190A1, which is herein incorporated by reference in its entirety. An affinity reagent may be formed with its valency increased by at least about 1, 2, 3, 4, 5, 6, 7, 8, 9, 10, or more than 10 paratopes. For example, a divalent antibody may be modified into a tetravalent antibody.

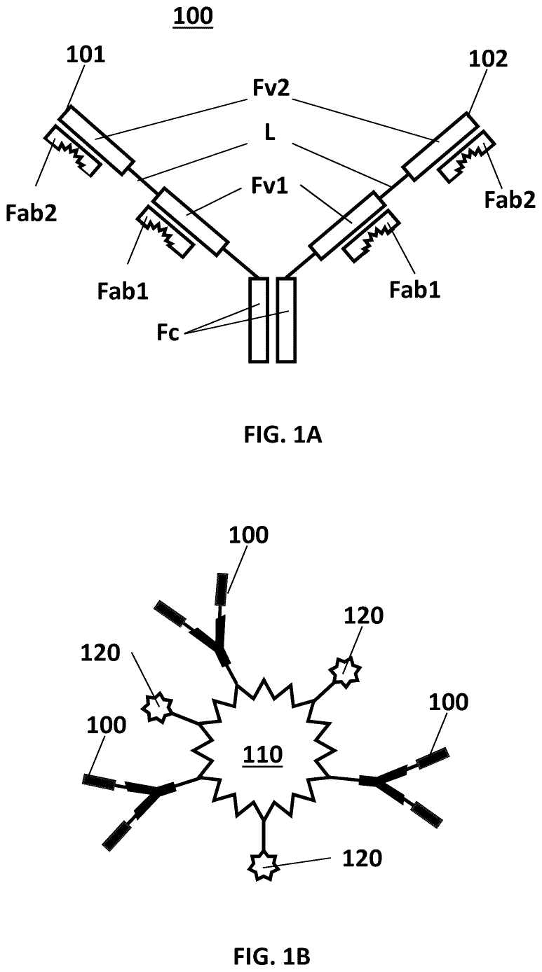

FIGS. 1A-1B depict aspects of binding reagents comprising engineered antibodies with increased valency. FIG. 1A illustrates a full antibody 100 comprising two heavy chains (101, 102). The heavy chains are joined at the crystallizable fragment portions Fc of the heavy chains 101 and 102. Each heavy chain further comprises a first variable fragment Fv1 and a second variable fragment Fv2. The first variable fragment Fv1 may be attached and separated from the second variable fragment Fv2 by a linker L (e.g., a poly-PQ linker, a poly-GQ linker, etc.). A first light chain Fab1 is bound to a first variable fragment Fv1 for each heavy chain, and a second light chain Fab2 is bound to a second variable fragment Fv2 for each heavy chain. Depending upon the type of antibody, each light chain (Fab1, Fab2) may form one or more paratopes when bound to a heavy chain. In some cases, Fab1 and Fab2 may comprise the same amino acid sequences, thereby producing substantially identical paratopes. Accordingly, the paratopes of Fab1 and Fab2 may have substantially the same binding specificity. In other cases, Fab1 and Fab2 may comprise the differing amino acid sequences, thereby producing differing paratopes. Accordingly, the paratopes of Fab1 and Fab2 may have differing binding specificities. Although the engineered antibody 100 is shown with one additional paratope-containing domain on each arm, additional paratope-containing domains can readily be added by attaching additional Fvs to the heavy chain.

Added domains of an engineered affinity reagent may be joined to the affinity reagent by a linker. A linker may comprise a residue sequence (e.g., a nucleotide sequence, an amino acid sequence). A linker may comprise at least about 1, 2, 3, 4, 5, 10, 15, 20, 25, 30, 40, 50, or more than 50 repeats of a residue sequence. Alternatively or additionally, a linker may comprise no more than about 50, 40, 30, 25, 20, 15, 10, 5, 4, 3, 2, or less than 2 repeats of a residue sequence. A linker may comprise a non-biological polymer (e.g., a PEG linker).

FIG. 1B depicts a binding reagent formed from one or more engineered antibodies 100 such as the engineered antibody of FIG. 1A. As shown, the binding reagent comprises 3 engineered antibodies 100 that are attached to a linking moiety, such as a particle 110 (e.g., a nucleic acid dendrimer, a polymer dendrimer, a dendron, a polymer scaffold, a nucleic acid nanoparticle, a quantum dot, etc.). Detectable labels 120 (e.g., fluorophores, luminophores, radiolabels, spin labels, nucleic acid barcodes, peptide barcodes, etc.) may also be attached to the linking moiety 110. Alternatively, detectable labels 120 may be attached to the engineered antibodies 100. The binding reagent may have an increased total valency due to the engineered antibodies 100. If the engineered antibodies 100 contain differing paratopes due to differing Fabs, the binding reagent may have increased valencies for each epitope or binding ligand recognized by the engineered antibodies.

A linking moiety may be useful for joining together multiple components of a binding reagent. Useful linking moiety compositions are described in U.S. Pat. No. 11,692,217, and U.S. Patent Publication Nos. 20230090454A1, 20240053333A1, 20240280568A1, 20240426839A1, each of which is incorporated by reference in its entirety. A linking moiety may be advantageous for joining a detectable label to one or more affinity reagents to form a binding reagent. In some configurations, a detectable label may be attached to the structure of a linking moiety of a binding reagent, in which no detectable labels are attached to the structure of an affinity reagent of the binding reagent.

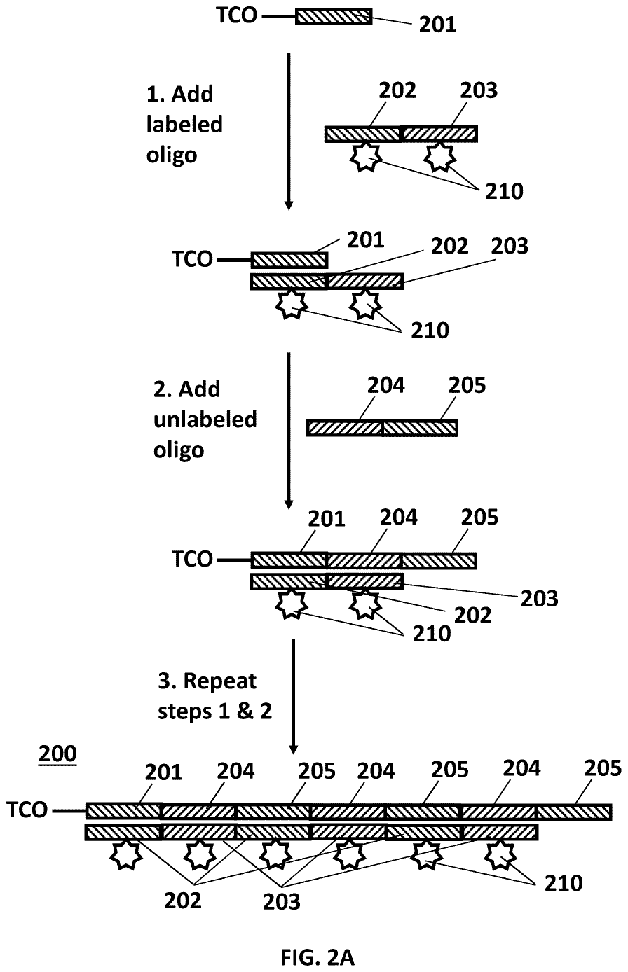

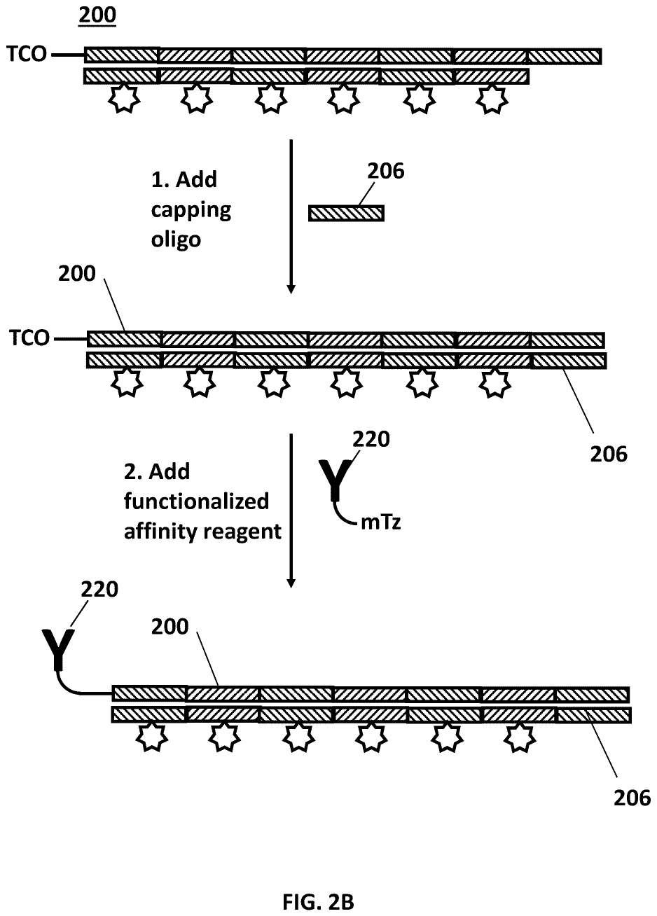

Certain types of linking moieties may be prone to a degree of size dispersity due to their formation methods. For example, dendrimeric particles may have some degree of branching dispersity. Such size dispersity may in turn produce dispersity in binding reagents that incorporate the linking moieties, for example due to incorporation of fewer or additional biding reagent components (e.g., affinity reagents, detectable labels, tether strands, etc.). FIGS. 2A-2B illustrate a method of forming a linking moiety that may be incorporated into a binding reagent. The method of formation may facilitate improved control over the formation process, thereby providing decreased dispersity in resultant binding reagents incorporating the linking moieties.

FIG. 2A depicts a sequence of steps for forming a linking moiety 200. Initially, a first oligonucleotide 201 containing a capture moiety is provided. As shown, the capture moiety is a click-type reagent (e.g., TCO), however the skilled person can readily envision other useful capture moieties (e.g., streptavidin/biotin, SpyCatcher/SpyTag, etc.) for attachment of entities to the linking moiety 200. In a first step, a second oligonucleotide is introduced. The second oligonucleotide comprises a first nucleotide sequence 202 that is complementary to the first oligonucleotide 201 and a second oligonucleotide sequence 203. The result of the first step can be a first intermediate nucleic acid formed by the hybridization of the first oligonucleotide 201 to the first nucleotide sequence 202 of the second oligonucleotide. The first intermediate nucleic acid can comprise a single-stranded sequence containing the second nucleotide sequence 203 of the second oligonucleotide. The second oligonucleotide may further comprise one or more attached detectable labels 210. Alternatively, the second oligonucleotide may incorporate at least one attachment site (e.g., a modified nucleotide) that facilitates attachment of a detectable label or other component to the linking moiety 200. In a second step, a third oligonucleotide is introduced. The third oligonucleotide comprises a first nucleotide sequence 204 that is complementary to the second nucleotide sequence 203 of the second oligonucleotide and a second nucleotide sequence 205 that is complementary to the first nucleotide sequence 202 of the second oligonucleotide. The second nucleotide sequence 205 of the third oligonucleotide may contain an identical nucleotide sequence to a nucleotide sequence of the first oligonucleotide 201. In some cases, the nucleotide sequence of the first oligonucleotide 201 may be identical to the nucleotide sequence of the second nucleotide sequence 205 of the third oligonucleotide. The result of the second step can be a second intermediate nucleic acid containing the third oligonucleotide hybridized to the first intermediate nucleic acid. The second intermediate nucleic acid can comprise a single-stranded sequence containing the second nucleotide sequence 205 of the third oligonucleotide. In a third step, the first and second step may be repeated sequentially, thereby forming a third intermediate nucleic acid containing another second oligonucleotide and third oligonucleotide. The third step may be repeated until the nucleic acid has achieved a desired size or length, thereby forming the linking moiety 200.

FIG. 2B depicts a method of incorporating the linking moiety 200 into a binding reagent. Optionally, the linking moiety 200 may be contacted with a capping oligonucleotide 206 that is complementary to a single-stranded portion of the linking moiety 200. The capping oligonucleotide may remove any remaining single-stranded nucleic acid from the linking moiety 200, thereby possibly increasing the stability of the linking moiety 200 and decreasing a likelihood of aggregation. The linking moiety may also be contacted with a ligation enzyme (not shown), thereby further stabilizing the structure of the linking moiety 200. The linking moiety 200 may be contacted with an affinity reagent 220. The affinity reagent 220 may comprise a complementary capture moiety that is configured to bind to the capture moiety of the linking moiety 200. As shown, the affinity reagent is attached to an methyltetrazine (mTz) functional group that can covalently bond to the transcyclooctene (TCO) functional group of the linking moiety 200. The linking moiety 200 can attach to the affinity reagent 220, thereby forming a binding reagent.

The skilled person will readily recognize numerous variations of the method of FIGS. 2A-2B. For example, the method can be modified to introduce the second oligonucleotide and third oligonucleotide simultaneously. This may be advantageous for reducing the number of steps and any necessary intermediate purification processes, but could result in increased dispersity of size or length of linking moieties 200, thereby causing dispersity in the quantity of detectable labels 210 incorporated into individual linking moieties 200. In another example, an oligonucleotide that contains a modified or functionalized nucleotide may be introduced. The modified or functionalized nucleotide can facilitate attachment of entities to the linking moiety 200. The attachment site can be used to branch the linking moiety 200, for example to form additional nucleic acid arms. Branching may facilitate attachment of additional binding reagent components (e.g., affinity reagents, detectable labels, tether strands, etc.). In another example, the third oligonucleotide may be labeled rather than the second oligonucleotide, or both the second and third oligonucleotides may be labeled with detectable labels 210.

It may be preferable to control the relative spacing and/or orientation of detectable labels (e.g., fluorophores, luminophores) or tether strands of a binding reagent. For example, to achieve the greatest amount of light emission from light-emitting labels, the light-emitting labels may need to be sufficiently separated to inhibit quenching effects. Certain types of linking moieties, such as nucleic acid nanoparticles, have the advantage of tunability, thereby facilitating attachment of detectable labels to the linking moiety at most optimal positions. Other types of linking moieties, such as dendrimeric polymers, dendrons, or branched polymers, may have attachment sites for attachment of moieties such as detectable labels that are occupied in a stochastic fashion. The challenge of controlling spacing and/or orientation of detectable labels may also occur when affinity reagents are directly labeled (i.e., attachment of detectable labels to the affinity reagent structure). Accordingly, such linking moieties may have non-optimal spatial separation and/or orientation of moieties such as detectable labels or tether strands. Provided herein are clustered moiety compositions that can facilitate spacing and orientation of moieties that are attached to a linking moiety or affinity reagent.

In an aspect, provided herein is a composition, comprising: a) an affinity reagent, and b) a polymer comprising at least two branches and an attachment moiety, wherein the attachment moiety is attached to the affinity reagent, and wherein each branch of the at least two branches is attached to a detectable label.

In another aspect, provided herein is a composition, comprising: a) a first polymer comprising two or more branches, wherein a first branch of the two or more branches is attached to an affinity reagent, and b) a second polymer comprising two or more branches, wherein the second polymer is attached to a second branch of the first polymer, and wherein each branch of the two or more branches of the second polymer is attached to a moiety (e.g., a detectable label, a tether strand). In some configurations, the first polymer may be a linking moiety (e.g., a dendrimer, a dendron, etc.).

In another aspect, provided herein is a composition comprising an affinity reagent attached to a clustered label composition by a coordination bond, wherein the clustered label composition comprises a polymer comprising at least two branches, wherein each of the at least two branches is attached to a moiety (e.g., a detectable label, a tether strand).

A clustered moiety composition may comprise a branched polymer. Exemplary branched polymers can include branched polyethylene glycol (PEG), branched polyglycerol, and branch polyerythritol. In some configurations, at least one branch or arm of a branched polymer may be attached to an attachment moiety that facilitates attachment of the clustered moiety composition to another entity (e.g., a linking moiety, an affinity reagent). Alternatively, a branched polymer may comprise an attachment moiety that is not attached to a branch of the branched polymer. A branched polymer may comprise at least about 2, 3, 4, 5, 6, 7, 8, 9, 10, 15, 20, 30, 40, 50, 100, or more than 100 branches or arms. Alternatively or additionally, a branched polymer may comprise no more than about 100, 50, 40, 30, 20, 15, 10, 9, 8, 7, 6, 5, 4, 3, or less than 3 branches or arms.

A branched polymer of a clustered moiety composition may be provided an attachment moiety that facilitates attachment of the clustered moiety composition to another entity (e.g., a linking moiety, an affinity reagent). In some configurations, an attachment moiety may be configured to form a covalent bond with a complementary attachment moiety of the affinity reagent. In particular configurations, an attachment moiety may form a covalent bond with a complementary attachment moiety of a linking moiety attached to the affinity reagent. An attachment moiety can comprise a Click-type reagent, a member of a receptor-ligand binding pair (e.g., an isopeptide bond-forming pair), a chelating moiety, or a ligand for a chelating moiety. In other configurations, an attachment moiety may form a non-covalent bond with a complementary attachment moiety of a linking moiety attached to the affinity reagent. In particular configurations, an attachment moiety may form a non-covalent bond with a complementary attachment moiety of a linking moiety attached to the affinity reagent. An attachment moiety can comprise a member of a receptor-ligand binding pair (e.g., streptavidin/biotin, avidin/biotin).

A binding reagent, as set forth herein, may comprise two or more clustered moiety compositions, as set forth herein. A binding reagent may comprise two or more clustered detectable label composition. A binding reagent may comprise a clustered label composition and a clustered tether strand composition. A binding reagent may comprise two or more clustered moiety compositions and two or more affinity reagents.

The present disclosure provides affinity reagents that are modified in a stochastic manner or modified in a site-specific manner. Clustered moiety compositions may be attached to affinity reagents in a stochastic manner. For example, affinity reagents may be attached to non-terminal residues of an affinity reagent (e.g., random lysines or cysteines of a protein affinity reagent). Clustered moiety compositions may be attached to attachment moieties of affinity reagents that have been provided in a stochastic manner. Clustered moiety compositions may be attached to affinity reagents in a site-specific manner. For example, affinity reagents may be attached to terminal residues of an affinity reagent (e.g., N-terminal or C-terminal amino acids of a protein affinity reagent). Clustered moiety compositions may be attached to attachment moieties of affinity reagents that have been provided in a site-specific manner.

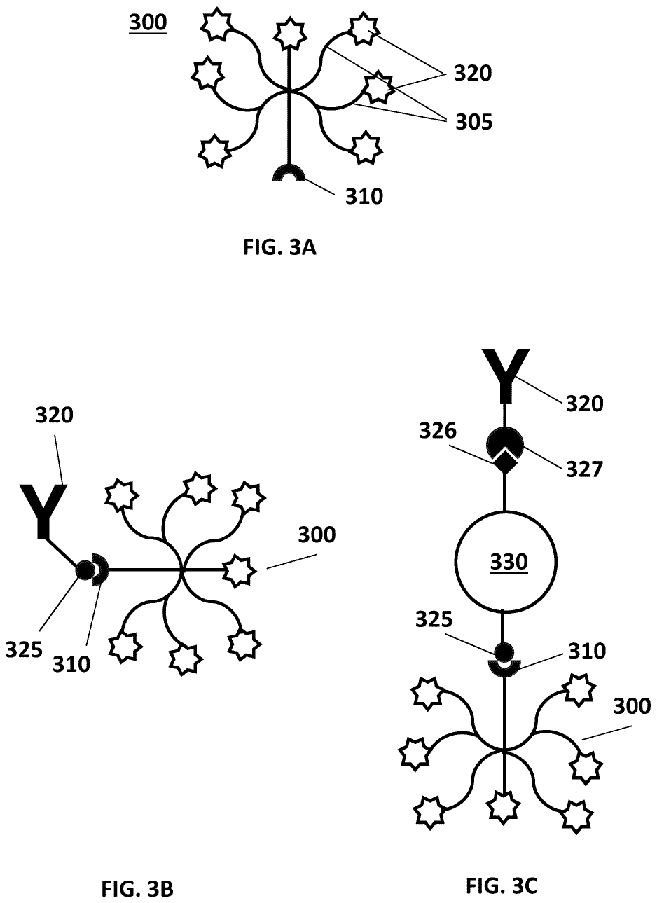

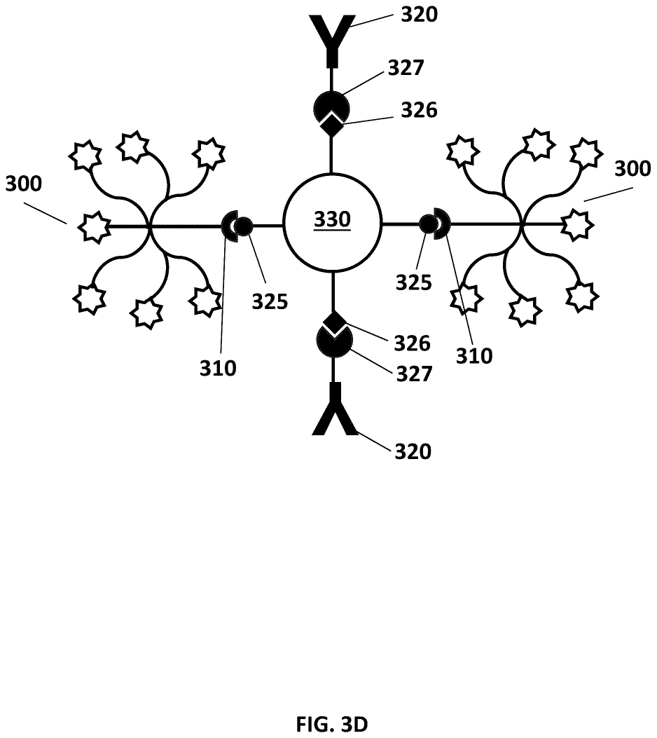

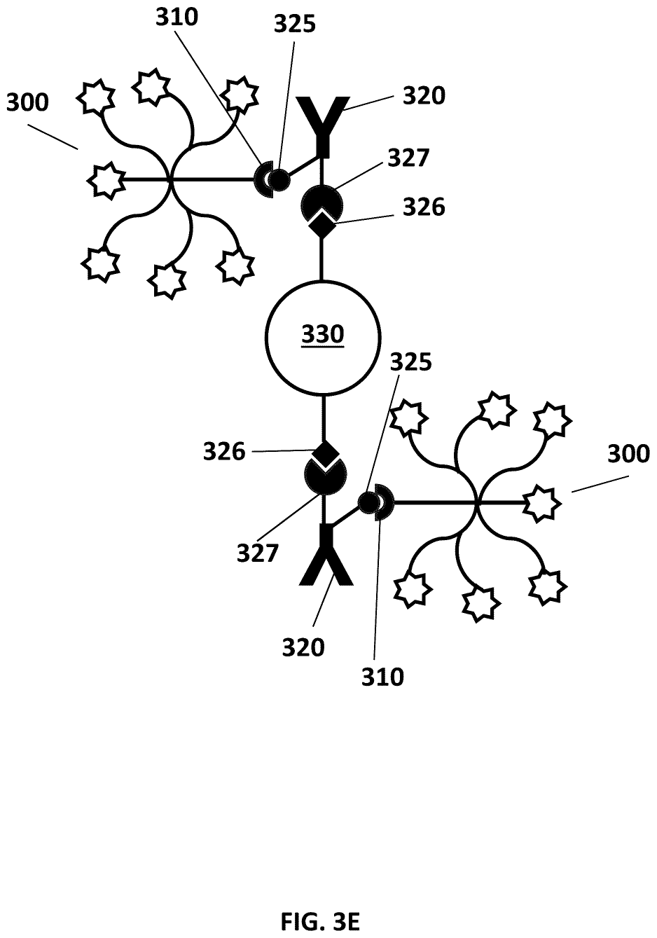

FIGS. 3A-3E depict compositions comprising clustered detectable labels. The compositions may be preferable for attaching light-emitting detectable labels to binding reagent compositions set forth herein. FIG. 3A depicts a clustered detectable label composition 300. The composition comprises a branched polymer comprising arms 305 with terminally attached detectable labels 320 (e.g., fluorophores, luminophores, barcodes, spin labels, radiolabels, etc.). The length of the polymeric arms 305 can be chosen to generate sufficient spacing between detectable labels 320 to inhibit interactions that are deleterious to detection (e.g., quenching, photon transfer effects, steric crowding, etc.). A clustered detectable label composition 300 may provide an average spacing of at least about 1 nanometer (nm), 2 nm, 3 nm, 4 nm, 5 nm, 10 nm, 15 nm, 20 nm, or more than 20 nm between nearest neighbor detectable labels 320. Alternatively or additionally, a clustered detectable label composition 300 may provide an average spacing of no more than about 20 nm, 15 nm, 10 nm, 5 nm, 4 nm, 3 nm, 2 nm, 1 nm, or less than 1 nm between nearest neighbor detectable labels 320. The clustered detectable label composition may further comprise an attachment moiety 310 that facilitates attachment of the composition to a linking moiety or affinity reagent. For example, the attachment moiety 310 may comprise a moiety of peptide residue, tag residue (e.g., SpyTag, sortase tag, Halo tag), nucleotide residue, or a reactive sidechain moiety or reactive functional moiety for click chemistry (e.g., aldehyde, HyNic, etc).

FIGS. 3B-3E illustrate binding reagent composition comprising the clustered detectable label composition 300 of FIG. 3A. FIG. 3B depicts attachment of the clustered detectable label composition 300 directly to the structure of an affinity reagent 320 via the attachment of the attachment moiety 310 to a complementary attachment moiety 325. The complementary attachment moiety 325 may be an endogenous moiety of the affinity reagent 320 (e.g., a terminal amino acid or nucleotide residue, tagging moieties such as those for SpyCatcher or sortase substrate, a reactive sidechain moiety or nucleotide base, etc.). Alternatively, the complementary attachment moiety 325 may be an exogenous moiety that is attached to the affinity reagent 320 (e.g., addition or alteration of a functional group, attachment of a peptide or nucleic acid tag, attachment of a binding ligand or receptor ligand, etc.). FIG. 3C depicts a binding reagent comprising a linking moiety 330 that joins the clustered detectable label composition 300 to an affinity reagent 320. The linking moiety comprises a first complementary attachment moiety 325 that reacts or binds to the attachment moiety 310 of the clustered detectable label composition 300, and a second complementary attachment moiety 326 that reacts or binds to an attachment moiety 327 of the affinity reagent 320.

FIG. 3D depicts a similar configuration to that of FIG. 3C, with additional components added to the binding reagent. As shown, the binding reagent comprises two affinity reagents 320 and two clustered dye compositions 300 attached to the linking moiety 300. Additional components, such as tether strands or additional affinity reagents or detectable labels, can readily be attached by providing additional attachment sites. It may be preferable to provide attachment sites with orthogonal attachment chemistries when attaching more than one type of component to a linking moiety. FIG. 3E illustrates a differing configuration of a binding reagent that combines elements of FIGS. 3A and 3D. The linking moiety 330 is attached to the affinity reagents 320 by binding of the complementary attachment moieties 326 to the attachment moieties 327 of the affinity reagents 320. Each affinity reagent further comprises a second complementary attachment moiety 325 that can couple to the attachment that binds to the attachment moiety 310 of a clustered detectable label composition 300.

A clustered detectable label composition may comprise a plurality of detectable labels attached to a scaffold molecule. A scaffold molecule may comprise a plurality of attachment sites that are individually configured to attach to a detectable label. The scaffold molecule may be configured to have at least about 2, 3, 4, 5, 6, 7, 8, 9, 10, 15, 20, 25, 30, 40, 50, 100, or more than 100 attachment sites. Alternatively or additionally, a scaffold molecule may be configured to have no more than about 100, 50, 40, 30, 25, 20, 15, 10, 9, 8, 7, 6, 5, 4, 3, or less than 3 attachment sites. After attaching detectable labels to a scaffold molecule, a clustered detectable label composition may comprise at least about 2, 3, 4, 5, 6, 7, 8, 9, 10, 15, 20, 25, 30, 40, 50, 100, or more than 100 detectable labels. Alternatively or additionally, a clustered detectable label composition may comprise no more than about 100, 50, 40, 30, 25, 20, 15 10, 9, 8, 7, 6, 5, 4, 3 or less than 3 detectable labels.

An affinity reagent may be modified for attachment of binding reagent components. Some affinity reagents, such as antibodies and derivatives thereof (e.g., antibody fragments), may contain numerous modifiable sites in their respective structures, including terminal amino acids, amino acid sidechains, and disulfide bridges. In some cases, an affinity reagent may be modified in a stochastic fashion if residues of the affinity reagent structure are modified randomly. For example, a pool of antibodies may be modified by a chemistry that attaches a moiety to random lysine, cysteine, or arginine sidechains of the antibody structure of the antibodies. Any two randomly chosen antibodies may be modified with a differing quantity of modifications and/or modifications at different lysine, cysteine, or arginine residues. In other cases, an affinity reagent may be modified in a site-directed or non-random manner if each affinity reagent of a pool of affinity reagents is modified at the same one or more residues of the affinity reagent structure. For example, a bridging group containing an attachment moiety may be inserted into a disulfide bridge that joins the heavy chains of an antibody, thereby providing an attachment site for other binding reagent components. In another example, an attachment moiety may be attached to a terminal residue of an antibody, thereby providing an attachment site for other binding reagent components.

Stochastic modification of affinity reagents may be less effective due to the possibility of modifying residues that are important to the binding property of the affinity reagents. For example, if an antibody is modified by random modification of lysine residues, it may be possible that a lysine residue in the CDR of the antibody is modified, thereby altering or inhibiting the binding of the antibody to a binding partner. The present disclosure provides a method of modifying an affinity reagent in a semi-stochastic fashion. The method may inhibit the modification of residues on portions of the affinity reagent that mediate binding interactions.

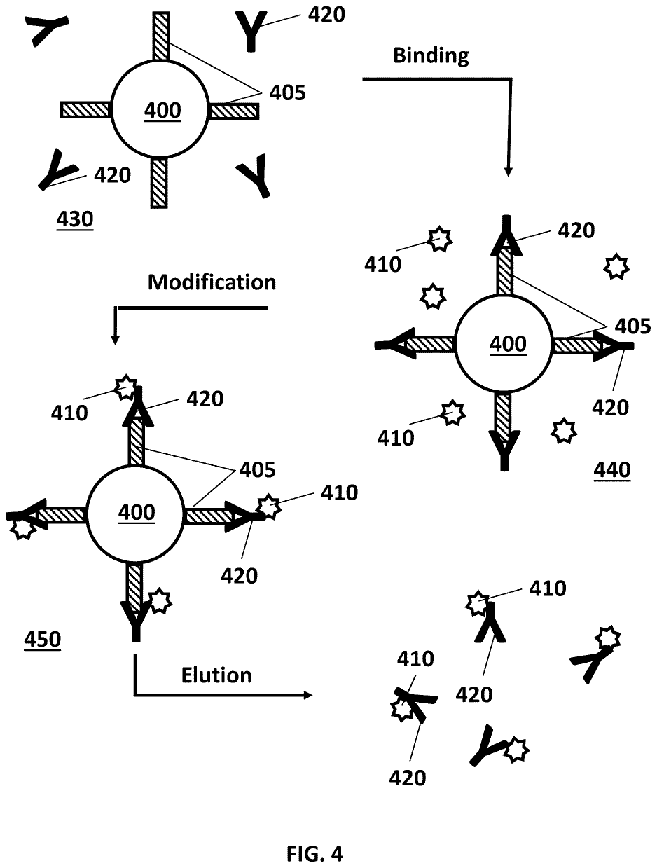

FIG. 4 illustrates steps of a method of modifying an affinity reagent in a semi-stochastic fashion. The upper left area of FIG. 4 depicts a first step, in which a solid support 400 (e.g., particles) are contacted with a plurality of affinity reagents 420. A plurality of binding partners 405 for the affinity reagents 420 (e.g., peptides, polypeptides, a binding epitope for the affinity reagents 420) is attached to the solid support 400. The contacting of the affinity reagents 420 to the solid support 400 may occur in the presence of a binding medium 430 that facilitates binding of the affinity reagents 420 to the binding partners 405. The first step provides a plurality of affinity reagents 420 bound to the solid support 400. Excess or unbound affinity reagents 420 may be separated from the solid support 400 prior to a second step. In a second step, the bound affinity reagents 420 are contacted with a modifying agent 410, optionally in the presence of a modification medium 440. The result of the second step is attachment of the modifying agents 410 to the affinity reagents 420 at available attachment sites (e.g., amino acid sidechains). In a third step, the modified affinity reagents 420 bound to the solid support 400 are eluted from the solid support 400, optionally in the presence of an elution medium 450 or other eluting condition (e.g., increased temperature). The method may produce a pool of affinity reagents that are modified in a semi-stochastic fashion but are not modified at the binding region of their structures.

Detectable probes set forth herein may be formed by attaching a detectable label to an affinity reagent in a site-specific or site-directed manner. In some cases, an affinity reagent may be provided with a terminal tag, such as a terminal peptide tag (e.g., a C-terminal peptide tag, an N-terminal peptide tag). Peptide tags can be ligated to an affinity reagent or produced by transgenic modification of a protein (e.g., antibodies expressed with the terminal peptide tag). In some cases, a terminal peptide tag can comprise a sortase tag. Accordingly, attaching a detectable label to an affinity reagent in a site-directed manner can comprise enzymatically attaching the detectable label to the terminal peptide tag of the antibody (e.g., via.a sortase-mediated transfer). In some cases, a terminal peptide tag can comprise an avi tag. Accordingly, attaching the detectable label to an affinity reagent in a site-directed manner can comprise: (i) enzymatically attaching a biotin moiety to the avi tag of the terminal peptide tag, and (ii) attaching a detectable label to the biotin moiety, wherein the detectable label further comprises a biotin-binding protein (e.g., streptavidin, avidin, neutravidin, etc.), and wherein the detectable label is attached to the biotin moiety by binding of the biotin-binding protein of the detectable label to the biotin moiety of the terminal peptide tag.

In some cases, a terminal peptide tag can comprise a polyhistidine tag. Accordingly, attaching a detectable label to an affinity reagent in a site-directed manner can comprise attaching the detectable label to the polyhistidine tag of the antibody by an imidazole-mediated chelation reaction (e.g., Ni-NTA mediated attachment).

In some cases, a detectable label further comprises a photo-catalyzed crosslinking moiety. Accordingly, attaching the detectable label to an affinity reagent in a site-directed manner can comprise: (i) combining the affinity reagent with the detectable label, (ii) contacting the affinity reagent and the detectable label with a light field, and (iii) in the presence of the light field, attaching the photo-catalyzed crosslinking moiety of the detectable label to the affinity reagent. An exemplary photo-mediated, site-directed attachment system is the oYo-Link system of AlphaThera. This system facilitates site-directed attachment of a linker to the Fc region of an antibody in the presence of ultraviolet light. A detectable label may comprise a binding moiety, wherein the binding moiety comprises the photo-catalyzed crosslinking moiety. Accordingly, attaching the detectable label to an affinity reagent in a site-directed manner can further comprise attaching the binding moiety to the antibody. In some cases, a photo-mediated attachment reaction may facilitate attachment of a moiety to a constant region of an antibody, or to a peptide tag attached to the antibody.

An antibody can be modified in a site-directed manner via insertion of a linking moiety into a disulfide bridge of the antibody. Such methods are described in U.S. Patent Publication No. 20240053333, which is herein incorporated by reference in its entirety. The linking moiety may provide for direct conjugation of a detectable label to an antibody, or may facilitate any other method of attachment set forth herein (e.g., enzymatic attachment, photo-mediated attachment, chelation attachment, etc.).

It may be preferable to provide a fluorescently-labeled binding reagent to a method set forth herein, in which the fluorescently-labeled binding reagent incorporates a quenching moiety. A quenching moiety may inhibit autofluorescence of fluorescent dyes, as can occur in systems utilizing single dye molecules or dye molecule pairs (e.g., FRET dye pairs). Preferably, a quenching moiety may be dissociable from a binding reagent, thereby facilitating removal of the quenching moiety when a binding reagent becomes bound to an analyte. A tether strand, as set forth herein, may be useful for incorporating fluorescent labels and/or quenching moieties.

In another aspect, provided herein is a method, comprising: a) binding an analyte to a binding reagent, wherein the binding reagent comprises a fluorescent dye molecule and a fluorescence quenching molecule, b) dissociating the fluorescence quenching molecule from the binding reagent, and c) after step b), detecting a signal from the fluorescent dye molecule of the binding reagent bound to the analyte.

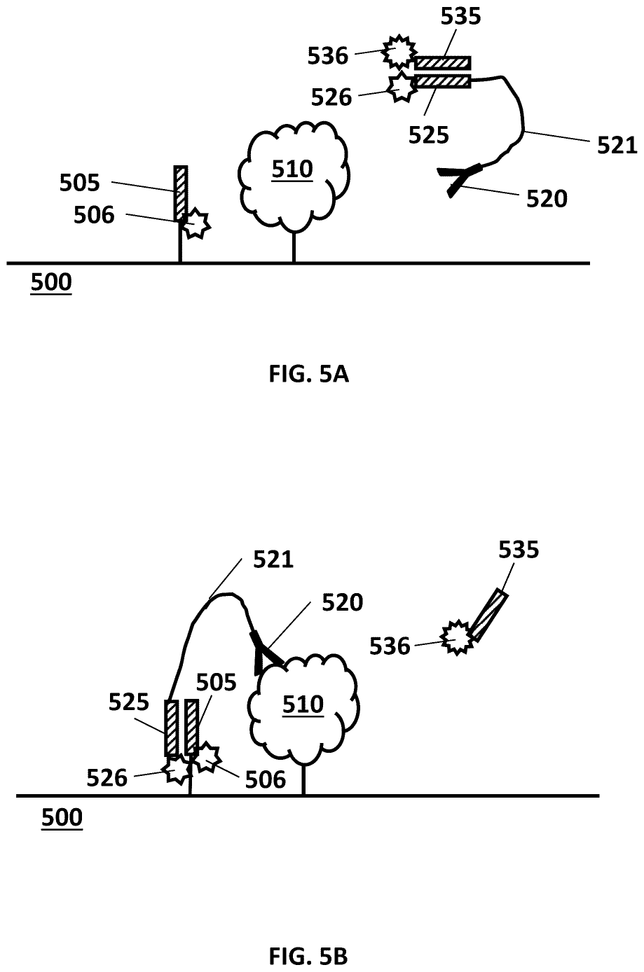

FIG. 5A-5B depict a binding reagent comprising a quenching moiety for a FRET-based detection system. FIG. 5A illustrates a solid support 500 containing a site with an immobilized analyte 510 and an immobilized docker strand comprising a first nucleotide sequence 505. The docker strand comprises a first FRET fluorescent dye 506. The solid support 500 is contacted with a binding reagent comprising an affinity reagent 520. The affinity reagent 520 is attached to a tether strand 521 comprising a second nucleotide sequence 525. The second oligonucleotide sequence 525 comprises a second FRET fluorescent dye 526. The binding reagent also comprises a third nucleotide sequence 535 that is hybridized to the nucleotide sequence 525. The third nucleotide sequence 535 comprises a quenching moiety 536. The quenching moiety 536 is located within a sufficient distance of the second FRET fluorescent dye 526 to inhibit fluorescence. FIG. 5B illustrates a second configuration of the system. The first nucleotide sequence 505 of the docker strand has displaced the third nucleotide sequence 535 from the second nucleotide sequence 525, thereby dissociating the quenching moiety 536. The first nucleotide sequence 505 hybridizes to the second nucleotide sequence 525, thereby bringing together the first FRET fluorescent dye 506 and the second FRET fluorescent dye 526. The presence of the binding reagent at the site of the solid support 500 may now be detected by stimulation of a FRET signal (e.g., by illumination of the solid support with light having an excitation wavelength of the FRET pair). The system depicted in FIGS. 5A-5B can be modified to provide the quenching moiety 536 on the docker strand (e.g., being displaced by the tether strand). Further, the depicted system is readily modified for non-FRET detection. For example, the tether strand can be attached to any fluorescent dye and the second fluorescent dye can be omitted from the docker strand.

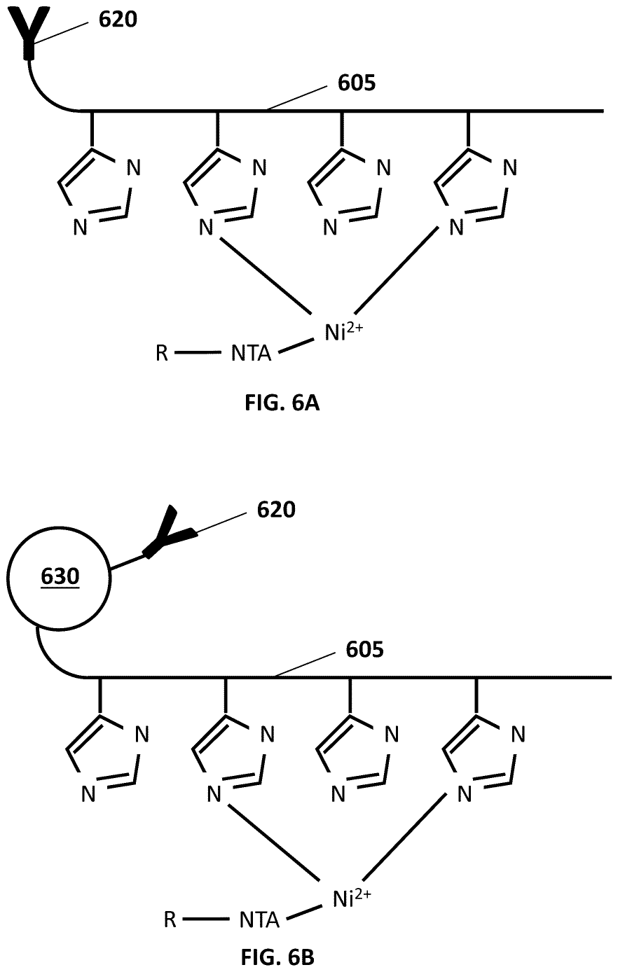

In some configurations, two or more components of a binding reagent may be attached to each other by chelation chemistry. Numerous biocompatible chelation chemistries are known, such as IMAC-like chelation, HyNIC chelation, and tetraamine chelation. Generally, a chelation-based attachment method will be provided a first binding reagent component comprising a chelating moiety and a second binding reagent component comprising a capture moiety that is configured to be bound by the chelating moiety. FIGS. 6A-6B formation of binding reagents or precursors thereof by a chelation method. The configurations are exemplified with an IMAC-based chelation chemistry (e.g., binding of nickel-nitrilotriacetic acid (Ni-NTA) to a polyhistidine moiety) but can readily be modified to utilize other chelation methods. FIG. 6A depicts an affinity reagent 620 that is attached to a polyhistidine moiety 605. The polyhistidine moiety 605 is bound by a second binding reagent component R, in which R is attached to a Ni-NTA chelating moiety. The second binding reagent component R could be any binding reagent component set forth herein, such as an affinity reagent, a detectable label, a clustered detectable label composition, or a tether strand. The configuration of FIG. 6A may be advantageous for incorporating commercial affinity reagents (e.g., his-tagged antibodies) into binding reagents. FIG. 6B depicts a second configuration, in which a linking moiety 630 is attached to an affinity reagent 620 and a polyhistidine moiety 605. The polyhistidine moiety 605 is utilized to attach an additional binding reagent component R to the linking moiety 630 by Ni-NTA chelation. The additional binding reagent component could be any binding reagent component set forth herein, such as an affinity reagent, a detectable label, a tether strand, a clustered detectable label composition, or a clustered tether strand composition.

A chelating moiety utilized for attachment of a component to a binding reagent may contain more than one chelated metal ion. Accordingly, a multivalent chelating moiety (i.e., a chelating moiety comprising more than one chelated metal ion) may form a more stable or stronger attachment due to an increased number of coordination bonds formed by the multivalent chelating moiety with a ligand. For example, Tris-NTA can chelate up to three nickel ions. Accordingly, Tris-NTA can form coordination bonds with up to six histidine groups of a polyhistidine moiety. Alternatively, a clustered moiety composition, as set forth herein, may comprise a plurality of chelating moieties. A component of a binding reagent may be attached by attachment of chelating moieties of the plurality of chelating moieties to a plurality of chelating ligands. A component of a binding reagent may be attached by attachment of chelating moieties of the plurality of chelating moieties to a single chelating ligand.

Some systems set forth herein utilize binding reagents comprising tether strands. A tether strand may facilitate retention of a binding reagent at a fixed spatial location (e.g., an array site containing an analyte) containing a complementary docker strand when the binding reagent is bound to a binding partner at the fixed spatial location. Aspects of tether strands and docker strands and systems that utilize them are discussed in U.S. Pat. No. 11,692,217, and U.S. Patent Publications 20240426839A1 and 20250066841A1, each of which is incorporated by reference in its entirety.

Some binding reagent configurations utilize tethers with differing binding chemistry from the binding chemistry of the binding reagent-analyte binding interaction. For example, an antibody-based affinity reagent may comprise an oligonucleotide tether strand that hybridizes to an oligonucleotide docker strand. Although the tether strand may facilitate retention of a binding reagent for sufficient time to detect the binding interaction of the binding reagent and analyte, the tether strand may complicate dissociation of the binding reagent from the analyte after a detection event. The optimal fluidic conditions for eluting the binding reagent from the analyte may not necessarily be optimal for dissociating the tether-docker interaction. The present disclosure provides binding reagent compositions that facilitate dissociation by incorporating a photolabile moiety into the tether strand or a photoisomerization moiety in the nucleotide base that can trigger oligonucleotide duplex dissociation upon light activation.

In another aspect, provided herein is a method, comprising: a) providing an analyte that is bound to an affinity reagent of a binding reagent, wherein the binding reagent further comprises a tether strand, wherein the tether strand comprises a photolabile moiety, and wherein the analyte is co-localized with a docker strand, b) binding the tether strand of the binding reagent to the docker strand, c) detecting the binding of the affinity reagent to the analyte, and d) after step c), contacting the photolabile moiety of the tether strand with light, thereby cleaving the photolabile moiety.

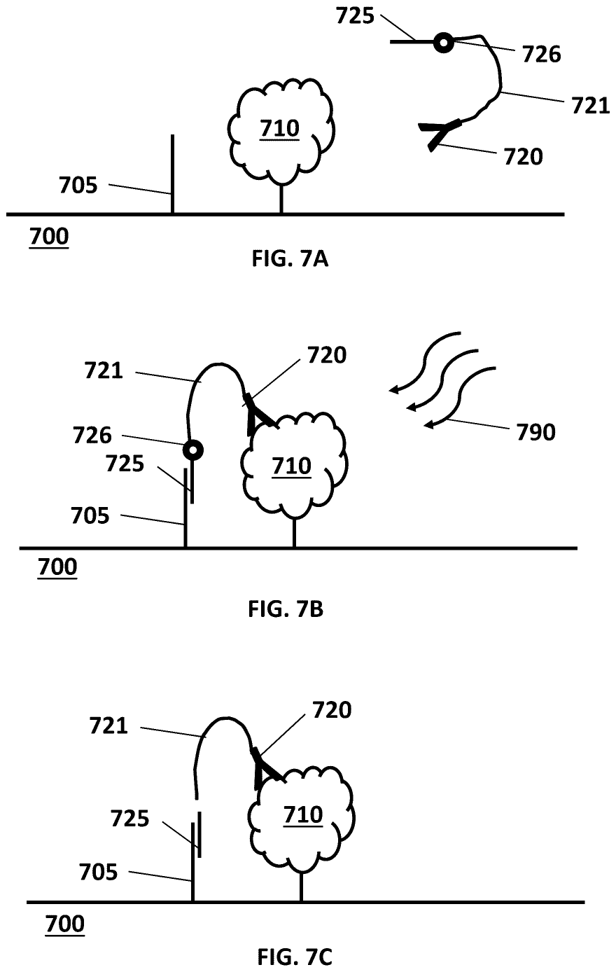

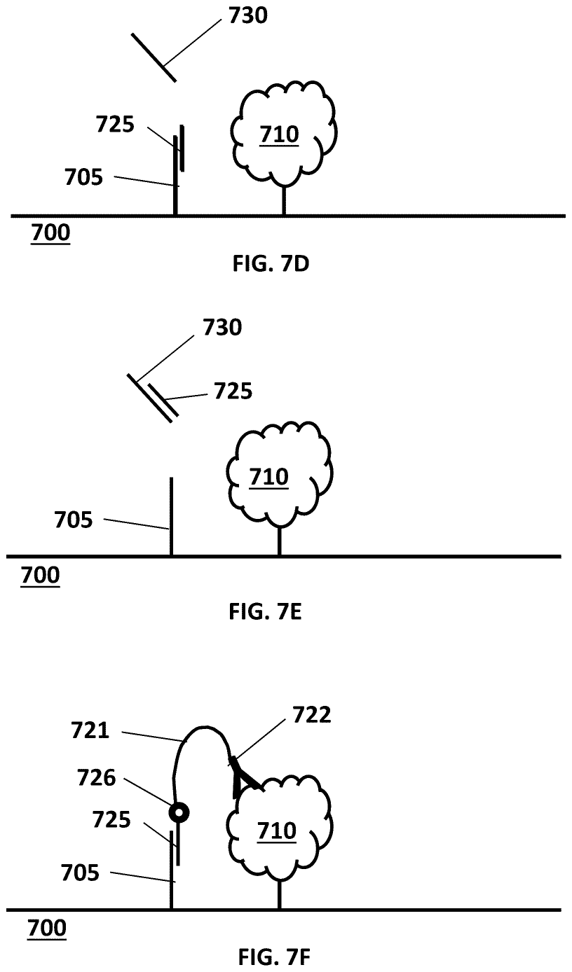

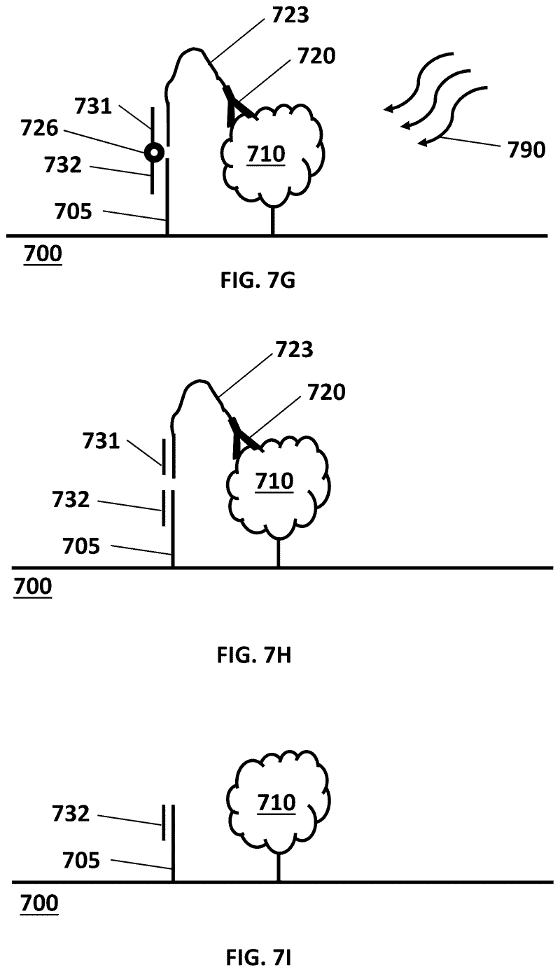

FIGS. 7A-7I illustrate systems that contain photolabile moieties in tether strands. In some cases, the photolabile group may be incorporated into the docker strand rather than the tether strand to produce a similar effect. FIG. 7A depicts a first configuration of a system comprising a solid support 700, solid support having a site containing an immobilized analyte 710 and a co-localized docker strand 705. The solid support is contacted with a binding reagent comprising an affinity reagent 720 attached to a tether strand 721. The tether strand 721 comprises a complementary moiety 725 that is configured to bind to the docker strand 705. The complementary moiety 725 is attached to the remaining tether strand 721 by a photolabile moiety 726. FIG. 7B depicts a second configuration of the system of FIG. 7A, in which the affinity reagent 720 has bound to the analyte 710, and the complementary moiety 725 of the tether strand 721 has bound to the docker strand 705. The binding interaction of the binding reagent with the analyte may be detected at the site of the solid support. Following a detection event, a sequence of dissociation steps may occur. First, the solid support 700 may be illuminated by light 790 of an excitation frequency for the photolabile moiety 726. The third configuration shown in FIG. 7C may occur after illumination of the solid support 700, in which absorption of a photon of light has cleaved the photolabile moiety 726, thereby separating a fragment of the tether strand 721 from the remaining tether strand of the binding reagent. Accordingly, the complementary moiety 725 is no longer attached to the tether strand 721 but remains coupled to the docker strand 705. In a subsequent dissociation step, the binding reagent may be eluted from the analyte 710 (e.g., by contacting the binding reagent and/or analyte with a dissociation medium). FIG. 7D depicts a fourth configuration of the system, in which a dissociation moiety 730 is contacted to the solid support 700. The dissociation moiety 730 may scavenge the complementary moiety 725 from the docker strand 705. The dissociation moiety 730 may be introduced in a same dissociation medium that is utilized to dissociate the binding reagent or may be introduced in a separate fluidic medium. The dissociation moiety 730 may displace (e.g., by toehold-mediated strand displacement) or competitively bind the complementary moiety 725, thereby dissociating it from the docker strand 705, as shown in FIG. 7E. Alternatively, it may be possible for the complementary moiety 725 of a subsequent binding reagent to competitively displace the complementary moiety 725 that is bound to the docker strand 705. Alternatively, it may be possible for the complementary moiety 725 to be dissociated by a dissociation medium, heating, pH change, or a combination thereof. FIG. 7F shows a subsequent configuration of the system, in which a new binding reagent comprising a different affinity reagent 722 is bound to the analyte 710. The sequence of steps depicted in FIGS. 7A-7E may be repeated for the new binding reagent.

FIGS. 7G-7I illustrate a similar system that utilizes a bridging moiety to join together a docker strand and a tether strand. The configuration depicted in FIG. 7G is similar to that of FIG. 7B, however the docker strand 705 is not directly coupled to the tether strand 723. Rather, a bridging moiety is coupled to the docker strand 705 and the tether strand 723, thereby coupling the docker strand 705 to the tether strand 723. The bridging moiety comprises a first complementary moiety 731 that is joined to a second complementary moiety 732 by a photolabile moiety 726. The first complementary moiety 731 is coupled to the tether strand 723, and the second complementary moiety 732 is coupled to the docker strand 705. The configuration of FIG. 7G may be illuminated with light 790 of an excitation frequency for the photolabile moiety 726. FIG. 7H depicts the system after illumination at the excitation wavelength of the photolabile moiety 726, thereby cleaving the photolabile moiety 726. FIG. 7I is analogous to FIG. 7D. Accordingly, similar methods may be utilized to dissociate the second complementary moiety 732 from the docker strand 705.

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g., the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. No. 10,473,654 or 11,282,585; U.S. Patent Publication Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g., amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e., observed binding events) and/or negative binding outcomes (i.e., observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g., positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654 or 11,282,585; U.S. Patent Publication Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g., the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US U.S. Patent Publication Nos. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g., technology developed by SomaLogic or Palamedrix) may also be useful for detecting proteins.

Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g., about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA.).

Non-limiting examples of functional groups for substituted phenyl isothiocyanate may include ligands (e.g., biotin and biotin analogs) for known receptors, labels such as luminophores, or reactive groups such as click functionalities (e.g., compositions having an azide or acetylene moiety). The functional group may be a DNA, RNA, peptide or small molecule barcode or other tag which may be further processed and/or detected.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase- or chemical-based ligation of the primer to a nucleic acid that is hybridized to the nucleic acid tag. The nucleic acid tag can be detected via hybridization to nucleic acid probes (e.g., in an array), amplification-based detections (e.g., PCR-based detection, or rolling circle amplification-based detection) or nuclei acid sequencing (e.g., cyclical reversible terminator methods, nanopore methods, or single molecule, real time detection methods). Exemplary methods that can be used for detecting proteins using nucleic acid tags are set forth in U.S. Patent Publication Nos. 2019/0145982 A1; 2020/0348308 A1; or 2020/0348307 A1, each of which is incorporated herein by reference.

In some configurations of the apparatus and methods set forth herein, one or more proteins can be detected on a solid support. For example, protein(s) can be attached to a solid support, the solid support can be contacted with detection agents (e.g., affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g., a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. No. 11,203,612 or 11,505,796 or U.S. Patent Publication No. 2023/0167488 A1, each of which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being at the same address in an array, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. No. 11,203,612 and 11,505,796, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g., single protein), a single complex of two or more molecules (e.g., a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

A method of the present disclosure can be performed in a multiplex format. In multiplexed configurations, different analytes can be attached to different unique identifiers (e.g., proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g., proteins) and/or affinity agents. The analytes and/or affinity agents can be attached to unique identifiers (e.g., addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g., ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g., an address in an array) using any of a variety of means. The attachment can be covalent or non-covalent. Exemplary covalent attachments include chemical linkers such as those achieved using click chemistry or other linkages known in the art or described in U.S. Pat. No. 11,203,612 or 11,505,796 or U.S. Patent Publication No. 2023/0167488 A1, each of which is incorporated herein by reference. Non-covalent attachment can be mediated by receptor-ligand interactions (e.g., (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in U.S. Pat. No. 11,203,612 or 11,505,796 or U.S. Patent Publication No. 2023/0167488 A1, each of which is incorporated herein by reference.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

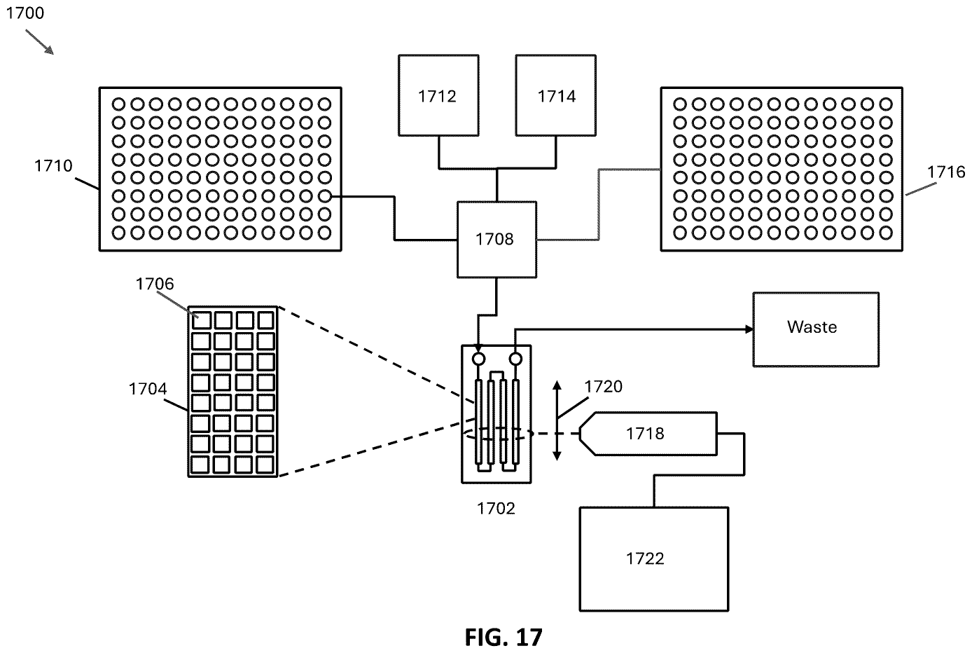

Additionally, provided herein are systems for performing the techniques, reagents, systems, and methods described herein. An example of a system is illustrated in FIG. 17. As shown, the system 1700 includes a flowcell 1702 that includes an array surface (shown as 1704) within the channels of the flow cell upon which individual protein molecules from a sample may be deposited and immobilized in locations 1706 that are individually addressable, and in particular cases are individually optically resolvable from each other using, e.g., fluorescence microscopy or scanning techniques.

The system will also typically include a fluidic delivery system 1708 that is configured to deliver different fluids to the flow cell 1702 through a series of fluidic lines and utilizing appropriate pumps, valves and other conventional fluid controls. The fluidics system 1708 may be fluidically coupled to various sources of fluids and reagents needed to carry out the analysis on the flow cell. For example, as shown, fluidic system 1708 is fluidly coupled to a source of a plurality of reagents 1710 (shown as a 96 well plate, although any number of different reagent storage systems of varying capacity may be employed) that includes a library of multiple affinity reagents that each have affinity for different characteristics of one or more proteins of interest. Additionally, fluidic system 1708 may also be coupled to sources of washing fluids or buffers 1712, and removal reagents 1714 (for removing bound affinity reagents following detection), as well as any other ancillary fluids and reagents needed for the analysis. Similarly, where flow cells are prepared on the system, the fluidic system may be coupled to sources of different sample materials that are to be analyzed 1716 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 1718, for detecting and recording fluorescent signals arising from different positions on the array surface. Such detection systems may generally include line scanning confocal fluorescent microscope systems, which are capable of scanning across large array surfaces (as shown by arrow 1720) to detect and record fluorescence across such surfaces at reasonably high scan rates.

The overall systems also typically include one or more computers or processors 1722 for controlling the operation of the instrument system including the fluidic system 1708 (e.g., to sample different sample sources 1716, reagent sources 1710 and delivery timing and volume of each), and detection system 1718, among other functions, and for recording the detected signals received from the detection system 1718, e.g., fluorescent signals, and analyzing such signals to identify potential binding by each of the different affinity reagents. Processors 1722 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been describe in, for example, U.S. Pat. Nos. 11,545,234, 10,473,654B1, and Egertson, et al., A theoretical framework for proteome-scale single-molecule protein identification using multi-affinity protein binding reagents, bioRxiv, Oct. 12, 2021, U.S. Patent Publication No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 1722 can be an electronic device of a detection system, the electronic device being integral to the detection system or remotely located with respect to the detection system. The computer system 1722 includes a computer processing unit (CPU, also “processor” and “computer processor” herein), which can be a single core or multi core processor, or a plurality of processors for parallel processing. The computer system 1722 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 1722 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 1722, can implement a peer-to-peer network, which may enable devices coupled to the computer system 1722 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 1722 can be included in the circuit. In some cases, the circuit is an application specific integrated circuit (ASIC).

The storage unit can store files, such as drivers, libraries and saved programs. The storage unit can store user data, e.g., user preferences and user programs. The computer system 1722 in some cases can include one or more additional data storage units that are external to the computer system 1722, such as located on a remote server that is in communication with the computer system 1722 through an intranet or the Internet.

The computer system 1722 can communicate with one or more remote computer systems through the network. For instance, the computer system 1722 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1722 via the network.

Methods as described herein can be implemented by way of machine (e.g., computer processor) executable code stored on an electronic storage location of the computer system 1722, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1722, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1722 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

It may be especially useful to provide an array of analytes with a diversity of polypeptide species. The diversity of polypeptide species may be measured with respect to a proteome, subproteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. No. 11,203,612 or 11,505,796; or U.S. Patent Publication No. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

An retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g., DNA or RNA), nucleotides (e.g., deoxyribonucleotides or ribonucleotides), nucleosides (e.g., deoxyribonucleosides or ribonucleosides), peptides (e.g., proteins, polypeptides or oligopeptides), amino acids, or sugars (e.g., saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40: 2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17): 3982 (2020); Zheng, et al. or Chem. Soc. Rev. 44, 4091-4130 (2015), each of which is incorporated herein by reference.

The present disclosure provides compositions and methods for improving binding of analytes to affinity reagents by increasing avidity of the binding interaction. In particular embodiments, avidity between an analyte and affinity reagent can be increased by association of a docker with the analyte and association of a tether with the affinity reagent. The docker and tether recognize each other and can thus bind to each other. Avidity of the interaction between the affinity reagent and analyte is a function not only of recognition between the paratope and epitope, but also recognition between the docker and tether.

A docker can be associated with an analyte via covalent and/or non-covalent attachment of the docker to the analyte. Similarly, a tether can be associated with an affinity reagent via covalent and/or non-covalent attachment of the docker to the affinity reagent. Exemplary attachment chemistries include those set forth herein in the context of attaching analytes and affinity reagents to retaining components, addresses of an array, solid supports, labels, etc. In some configurations, a docker or tether can be attached to a particle (e.g., structured nucleic acid particle), unique identifier, address or solid support to which an analyte or affinity reagent, respectively, is attached.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes a step of detecting association of the affinity reagent with the analyte, thereby identifying the analyte. In another option, the analyte is present in a sample including other analytes and the method further includes a step of separating the analyte from the other analytes via the association of the affinity reagent with the analyte.

The compositions and methods of the present disclosure are particularly well suited for detecting analytes using affinity reagents in non-equilibrium conditions. A typical binding assay employ an excess amount of affinity reagent and immobilized analytes to drive formation of an immobilized complex between the affinity reagent and analyte. In some assays the excess labeled affinity reagent in solution produces unwanted background that overwhelms signal produced by immobilized complexes. Removal of excess affinity reagents from solution creates a non-equilibrium condition that drives affinity reagents to dissociate from the immobilized analytes. The use of tethers and dockers can increase the half-life of the complexes under non-equilibrium conditions, thereby improving detectability of analyte-affinity reagent complexes.

A variety of different types of dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker type and tether type.

A docker can be any molecule or moiety that is capable of binding to a tether and a tether can be any molecule or moiety that is capable of binding to a docker. A particularly useful docker or tether is a nucleic acid strand having a nucleotide sequence that complements a nucleotide sequences of a tether or docker, respectively. A nucleic acid strand that is used as a docker or tether can include a sequence of at least 3, 4, 5, 6, 7, 8, 9, 10, 12, 14, 16, 18, 20, 25 or more nucleotides. Alternatively or additionally, a nucleic acid strand that is used as a docker or tether can include a sequence of at most 25, 20, 18, 16, 14, 12, 10, 9, 8, 7, 6, 5, 4, 3 or fewer nucleotides. Other useful dockers or tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers or tethers include, but are not limited to, an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker or tether can be a protein that recognizes a nucleic acid sequence such as a DNA binding protein or RNA binding protein. Exemplary nucleic acid-binding proteins, which can be used as dockers or tethers, and the nucleic acid moieties to which they bind, which can be used as tethers or dockers, respectively, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See, Leung et al. Nucleic Acids Res. 47(Database issue): D322-D329 (2019), which is incorporated herein by reference.

A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope.

Of course, a binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tm) of the duplex formed by the complementary sequences. The length of the complementary sequences can be at least 3, 4, 5, 6, 7, 8, 9, 10, 12, 14, 16, 18, 20, 25 or more nucleotides. Alternatively or additionally, the length of the complementary sequences can be at most 25, 20, 18, 16, 14, 12, 10, 9, 8, 7, 6, 5, 4, 3 or fewer nucleotides. The GC content of the complementary sequences can be at least 25%, 40%, 50%, 60%, 75%, or higher. Alternatively or additionally, the GC content of the complementary sequences can be at most 75%, 60%, 50%, 40%, 25% or lower.

Multiplex methods, in which a plurality of different analytes are processed in parallel, can employ universal dockers. The dockers are referred to as ‘universal’ because they are identical with respect to structural features that interact with tethers. For example, an array can include a plurality of addresses, each of the addresses being attached to an analyte that differs from other analytes in the array and each of the addresses being attached to a docker that is the same as other dockers in the array. A plurality of different analytes that are associated with universal dockers can be contacted with a plurality of different affinity reagents that are associated with tethers. Some or all the different affinity reagents can have the same tether structure. As such, the avidity effect of the dockers and tethers can be substantially uniform.

Methods that employ multiple different affinity reagents can employ universal tethers. The tethers are referred to as ‘universal’ because they are identical with respect to structural features that interact with dockers. For example, an array of analytes can be contacted with a plurality of different affinity reagents, each of the affinity reagents having a paratope that differs from other affinity reagents in the plurality and each of the affinity reagents being attached to a tether that is the same as other tethers in the plurality. The different affinity reagents can be present in a mixture that is simultaneously in contact with the array or, alternatively, the different affinity reagents can be serially contacted with the array.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. No. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g., organelles). For example, a sample can be derived from a tissue biopsy, biological fluid (e.g., blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g., an individual human), single tissue, single cell, single organelle (e.g., endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 μg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g., cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See, Ho et al., Cell Systems (2018), doi: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See, Aebersold et al., Nat. Chem. Biol., 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants are included. See, Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See, Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See, Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g., sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PUP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g., histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylglucosaminidase can be used to cleave non-reducing terminal β-linked N-acetylglucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

### EXAMPLES

### Example 1. Synthesis of Dendrimeric Binding Reagent

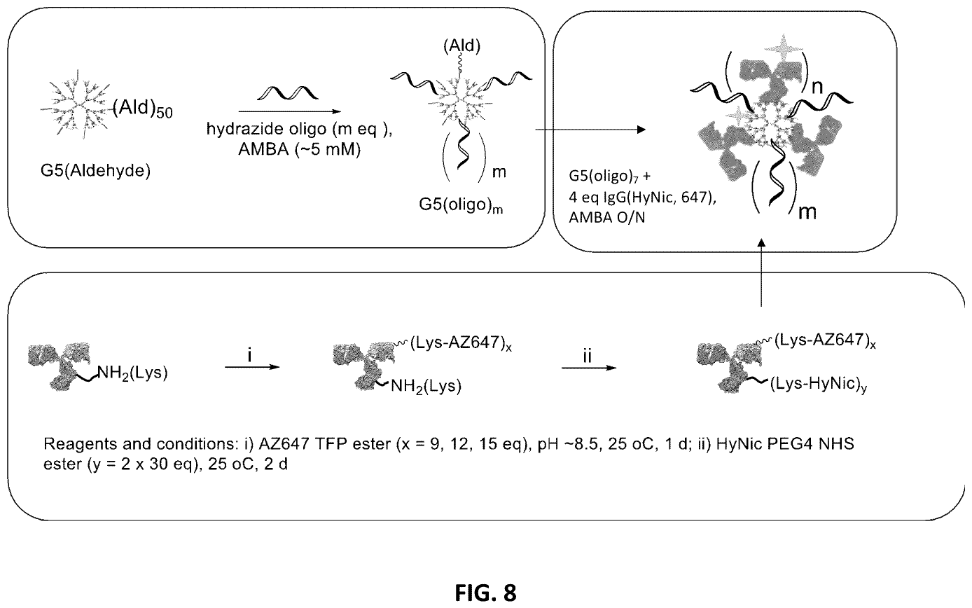

Affinity reagents comprising a plurality of antibodies attached to a dendrimeric linking moiety were synthesized according to the method presented in FIG. 8. In a first process, IgG antibodies were labeled stochastically with AZ647 fluorescent dye molecules. The AZ647 dyes were provided in a molar excess of 9×, 12×, or 15× relative to the molar quantity of antibodies to be labeled. The AZ647 dye molecules were attached to random lysine sidechains by reaction of sidechain amine groups with tetrafluorophenyl (TFP) ester functional groups of the AZ647 dye molecules. The labeling reaction was carried out at about pH 8.2 at 25° C. for 24 hours. After the labeling reaction, the antibodies were provided with hydrazinonicotinic acid (HyNIC) chelating groups for attachment to dendrimeric polymers. HyNIC chelating groups were attached by reaction of HyNIC-PEG4-N-hydroxysuccinimide (NHS) molecules with lysine sidechains. The HyNIC modification was performed at 25° C. for 48 hours.

Dendrimeric particles were prepared for the attachment of the stochastically labeled antibodies. Generation 5 dendrimers were provided with an average of 50 terminal aldehyde functional groups per dendrimer molecule, while the remaining terminal functional groups being primary amines were treated with glutaric anhydride for their modification to glutaramic acids. Aldehyde moieties of the G5(Aldehyde) dendrimer molecules were reacted with hydrazide-terminated tether oligonucleotides in media supplemented with 5 mM 2-amino-5-methoxybenzoic acid (AMBA) as a catalyst. After attachment of m equivalent (e.g., m=1, 2, 3, 4, 5, etc.) of the tether oligonucleotides to form G5(Oligo)ₘ particles, the G5(Oligo)ₘ were reacted with AZ647 dye and HyNic co-labeled antibodies in a 4× molar excess relative to the G5(Oligo)ₘ particles. The HyNIC groups of the antibodies were reacted covalently with the accessible aldehyde functional groups of the G5(Oligo)ₘ particles to cross-link the labeled antibodies to the G5(Oligo)ₘ particles. The resultant binding reagents comprised attached AZ647 dye-labeled antibody molecules and tether oligonucleotides in an n:m ratio.

### Example 2. Synthesis of Dendrimeric Binding Reagent with Clustered Dyes

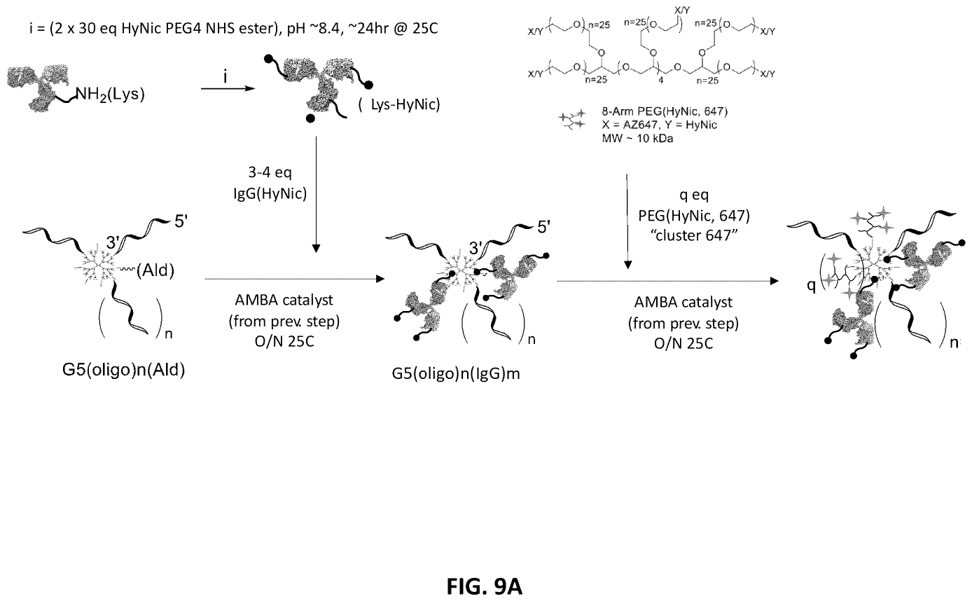

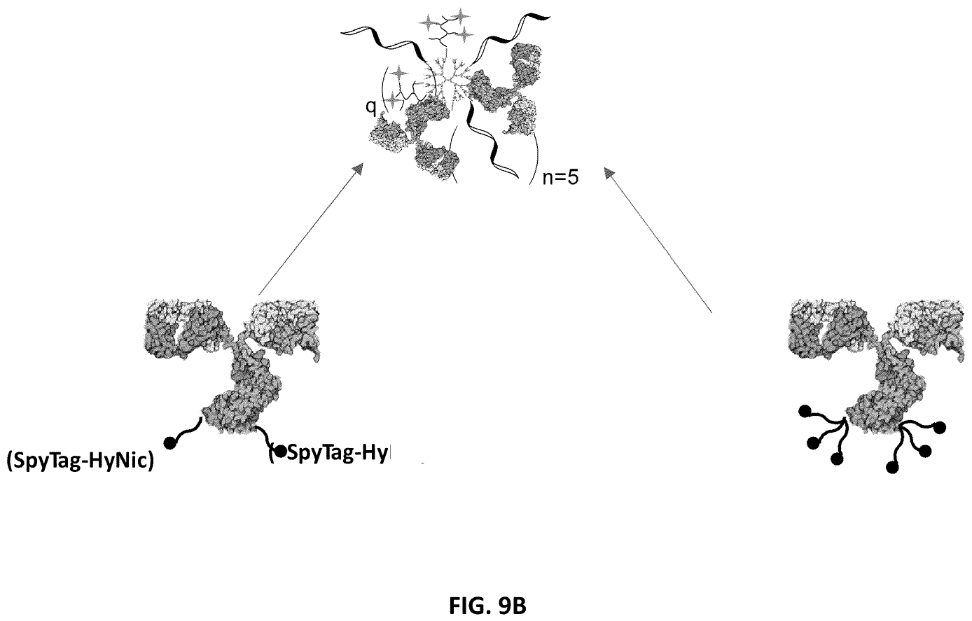

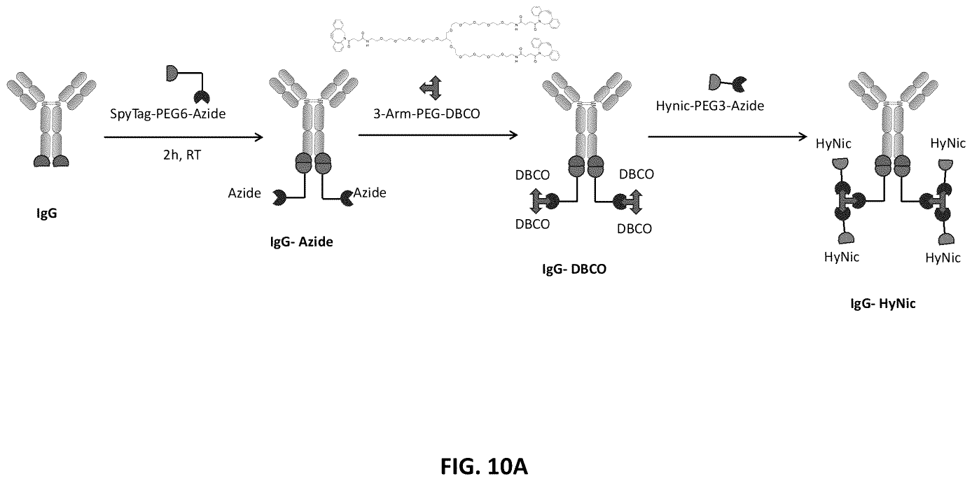

FIG. 9A provides a diagram of a method of forming a binding reagent comprising a dendrimeric linking moiety. The binding reagent is formed similarly to the method provided in Example 1, with the stochastic labeling of the antibodies with fluorescent dyes omitted from the process. Accordingly, after reacting the HyNIC-modified antibodies with the G5(oligo)ₙ(Aldehyde) particles, unlabeled G5(oligo)ₙ(IgG)ₘ particles are formed. Clustered fluorescent dyes are formed by covalently attaching AZ647 dyes to terminal functional groups of an 8-armed branched PEG molecule. Preferably, at least one arm of the branched PEG molecule is modified with a HyNIC chelating moiety. After forming the clustered AZ647 dye molecules, the binding reagent precursor is mixed with the clustered AZ647 dye composition in the presence of AMBA catalyst at 25° C. for 24 hours. The clustered dye molecules covalently attach to available aldehyde groups of the dendrimer molecule, thereby attaching the cluster dyes to the complex to form G5(oligo)ₙ(IgG)ₘ(AZ647)_{q} particles.

FIG. 9B illustrates an alternative method of forming the dendrimer binding reagent of FIG. 9A by utilizing antibodies that are modified in a site-specific manner. For the HyNIC chelation attachment chemistry described in FIG. 9A, the antibodies can be modified at a common conserved moiety, such as a SpyCatcher domain near a C-terminal amino acid on the crystallizable fragments of the IgG molecule. The left configuration shows an antibody with a single HyNIC functional group attached through the site-selective method of SpyCatcher-SpyTag conjugation to the C-terminus of each antibody heavy chain. The right configuration shows an antibody with multiple HyNIC functional groups attached in a similar method to the C-terminus of each antibody heavy chain. Additional attachment moieties provided in a site-specific manner may increase the likelihood that an antibody is incorporated into a binding reagent at a higher conjugation efficiency.

### Example 3. Formation of Multidentate Affinity Reagents

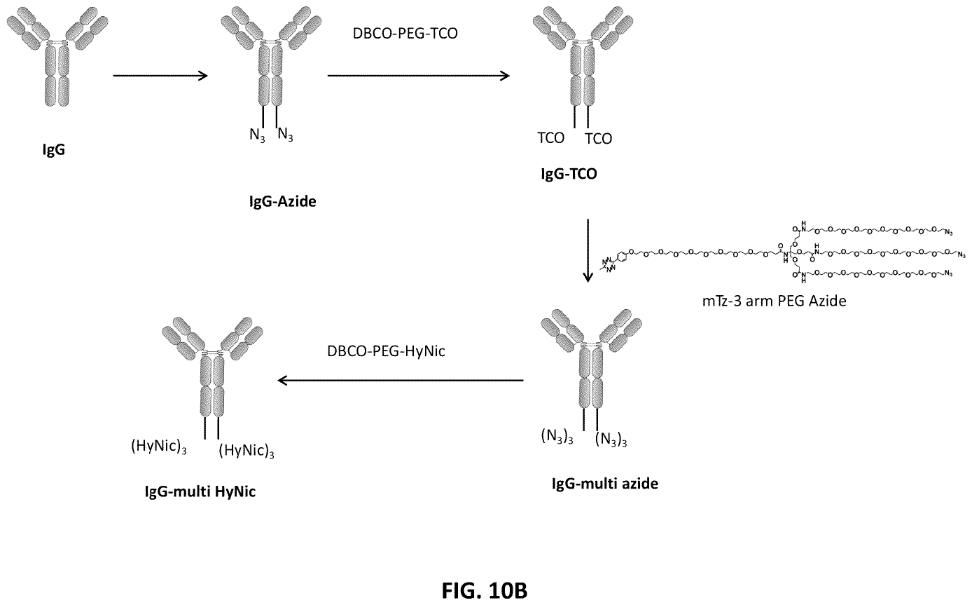

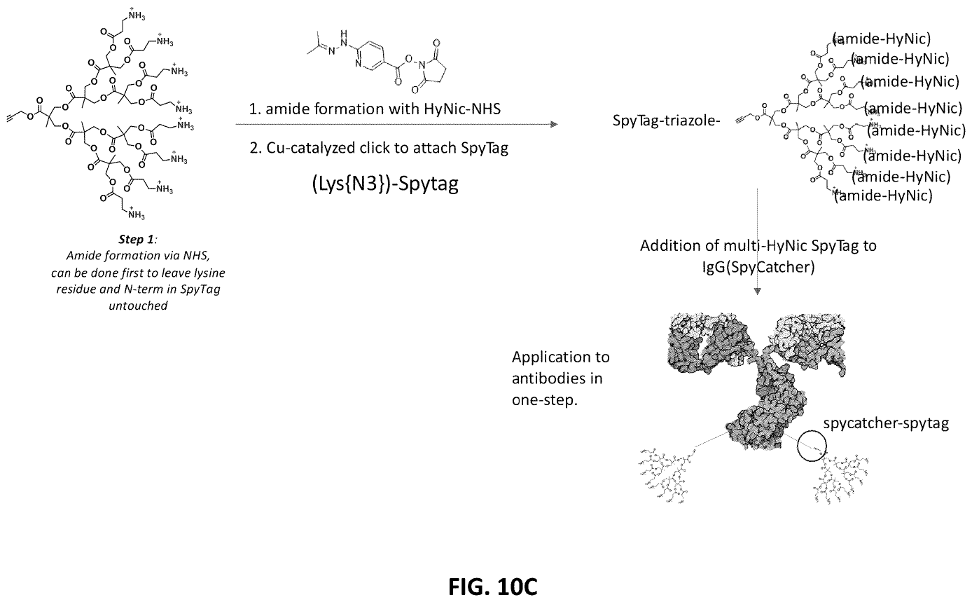

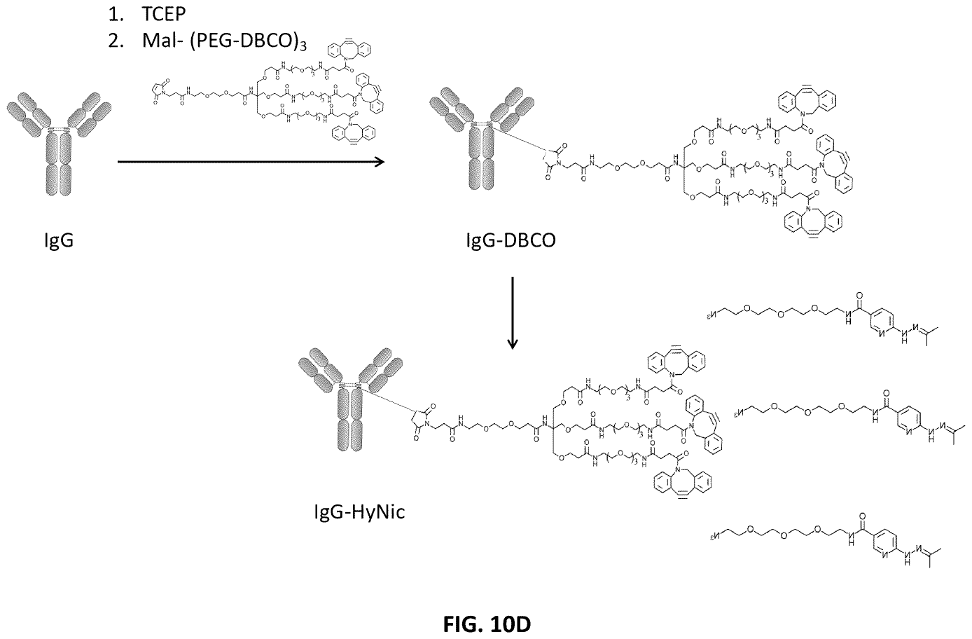

FIGS. 10A-10D depict reaction schemes for forming affinity reagents having multiple clustered attachment moieties that facilitate attachment of the affinity reagents to linking moieties. FIG. 10A depicts a scheme for modifying SpyCatcher-modified IgG antibodies. The site specifically-modified IgG antibodies are contacted with SpyTag-PEG-Azide molecules at 20° C. for 2 hours. The resultant azide-modified IgG antibodies are contacted with 3-armed PEG-dibenzocyclooctylene (DBCO) molecules. The PEG-DBCO molecules can react with the azide moieties of the IgG antibodies to form IgG(DBCO)₄ molecules. The DBCO-modified antibodies are next reacted with HyNIC-PEG-azide molecules to form IgG(HyNIC)₄ molecules. Each heavy chain has a site-specifically attached, branched polymer with two available HyNIC moieties that are configured to attach to a linking moiety composition set forth herein.

FIG. 10B depicts a scheme for modifying unmodified IgG antibodies. The unmodified IgG antibodies are site-specifically or stochastically modified with azide functional groups. The IgG(azide)ₙ molecules are then reacted with DBCO-PEG-TCO molecules to provide IgG(TCO)ₙ molecules. The IgG(TCO)ₙ molecules are next reacted with a four-branch PEG polymer having a single methyltetrazine (mTz) functional group and 3 terminal azide moieties, thereby forming IgG(N₃)₃ₙ molecules. The azide-modified antibodies can be reacted with DBCO-PEG-HyNIC molecules, thereby forming IgG(HyNIC)₃ₙ molecules that are configured to attach to a linking moiety composition set forth herein.

FIG. 10C depicts a scheme for attaching a dendron-like molecule to an IgG antibody. The dendron molecules have a single terminal alkynyl functional group and multiple terminal primary amines, depending upon the degree of branching of the dendron-like molecule. The dendron-like molecule is first reacted with HyNIC-NHS ester molecules to attach terminal HyNIC groups via amide bond formation between the primary amines and NHS esters. Next, the alkynyl group remaining in this HyNIC-modified dendron-like molecule is reacted with an azide(Lys)-terminated SpyTag peptide, forming a triazole linkage via a copper-catalyzed Click-type reaction. The resulting products are dendron-like molecules having a single SpyTag peptide and a plurality of HyNIC functional groups. The SpyTag-modified dendron-like molecule can be reacted with SpyCatcher-modified IgG antibodies to covalently attach the dendron-like molecules to the IgG antibodies.

FIG. 10D depicts an alternative site-specific method for attaching a cluster of attachment moieties to an antibody. The attachment site for the cluster is a disulfide linkage at the hinge region of the antibody where the heavy chains are attached. The antibody is partially reduced by TCEP, thereby producing free cysteine thiol functional groups due to disruption of disulfide bridges. A branched PEG molecule with a single maleimide functional groups and a plurality of DBCO functional groups is reacted to the reduced antibody by a maleimide-thiol reaction. After attaching the branched PEG to the antibody hinge, the DBCO molecules are reacted with azide-terminated HyNIC functional groups, thereby providing the multidentate HyNIC-modified antibody.

### Example 4. Synthesis of Dendrimeric Binding Reagent

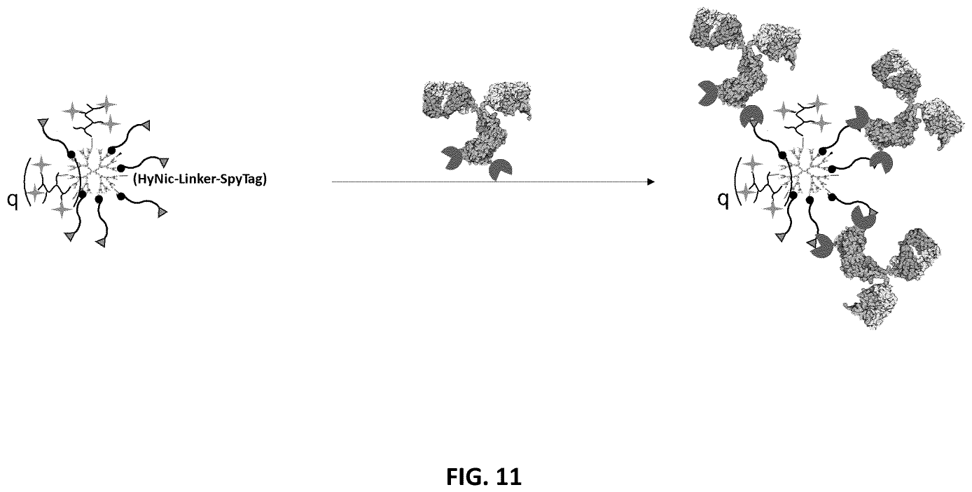

FIG. 11 illustrates a reaction scheme for forming a dendrimeric binding reagent. G5 (Aldehyde) particles are synthesized as described in Example 2. Before attachment of antibodies, clustered dyes are attached to available aldehyde groups by HyNIC chemistry, as described in Example 2 to form G5(Aldehyde)(cluster AZ647)_{q} particles. Remaining non-attached aldehyde groups are reacted with a HyNIC-PEG-SpyTag molecules to form G5(SpyTag)ₘ(cluster AZ647)_{q} particles. IgG antibodies with site-specific attachment of SpyCatcher protein are contacted with the G5(SpyTag)ₘ(cluster AZ647)_{q} particles, thereby forming G5(IgG)ₘ(cluster AZ647)_{q} particles via covalent attachment of the IgG-coupled SpyCatcher proteins to the particle-linked SpyTag peptides. This method may be modified to attach other moieties, such as oligonucleotides to dendrimer molecules.

### Example 5. Synthesis of Dendrimeric Binding Reagent with Fluorescent Protein A

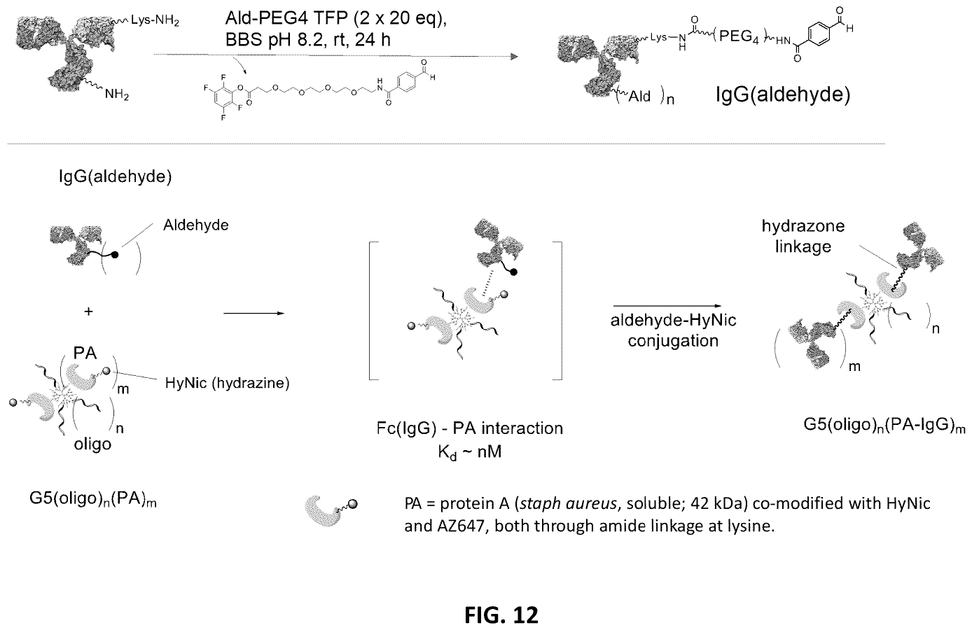

FIG. 12 provides a reaction scheme for forming a binding reagent comprising antibodies and a dendrimeric linking moiety. Antibodies are stochastically labeled with aldehyde functional groups by reacting available lysine sidechains of the IgG antibodies with aldehyde-PEG4-tetrafluorophenyl (TFP) molecules in borate buffered saline (BBS) solution at pH ~8.2, 20° C. for 24 hours. Dendrimer linking moieties can be provided in a similar method to the method of Example 1, with the antibodies substituted with fluorescently-labeled Protein A. Each attached fluorescently-labeled Protein A molecule further comprises a HyNIC functional group. The aldehyde-modified IgG antibodies are contacted to the G5(oligo)ₙ(PA)ₘ particles, thereby non-covalently binding IgG antibodies to the protein A molecules. After IgG molecules are bound to protein A molecules, the HyNIC functional groups of the protein A molecules can cross-link to the aldehyde groups of the antibodies, thereby covalently attaching them to the dendrimeric linking moiety.

### Example 6. Synthesis of Binding Reagent with Phiton Label

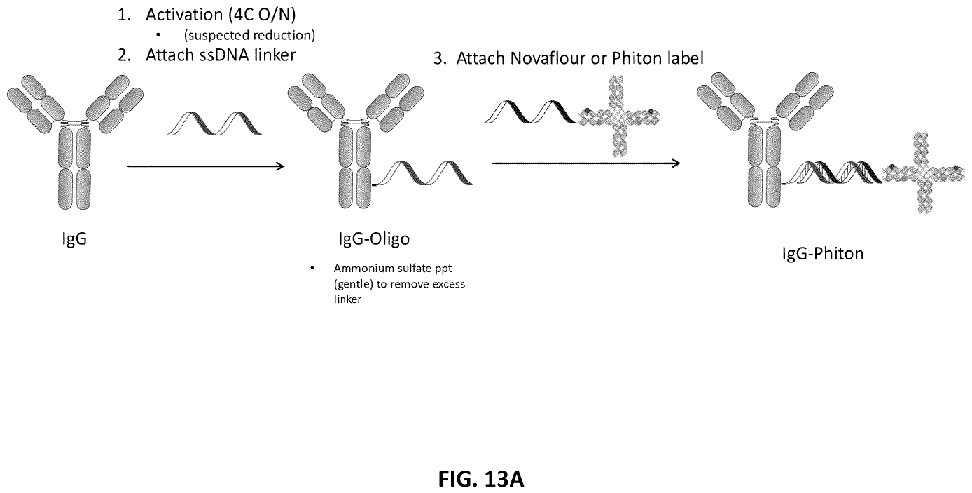

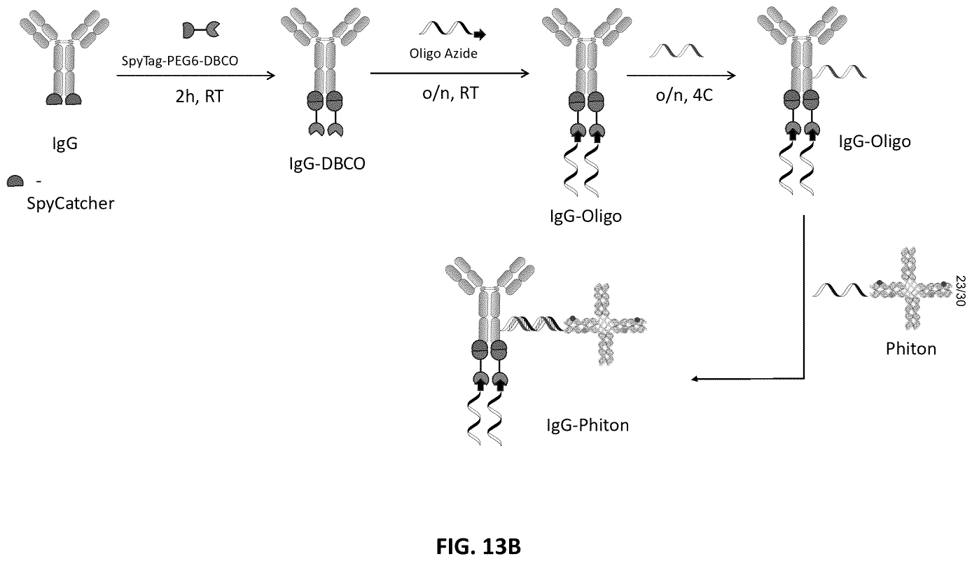

FIGS. 13A-13B illustrate reactive pathways for forming binding reagents utilizing affinity reagents labeled with fluorescent Phiton nucleic acid labels. FIG. 13A depicts a scheme for attaching a Phiton label to an antibody. An antibody can be modified in a site-specific manner or a stochastic manner to provide an antibody attached to single-stranded oligonucleotide. Excess oligonucleotide can be removed by gentle aluminum sulfate precipitation. After attaching the oligonucleotide to the antibody, the antibody can be contacted to a Phiton fluorescent label, thereby hybridizing a portion of the Phiton nucleic acid particle to the single-stranded nucleic acid of the antibody.

FIG. 13B shows a reaction scheme similar to the scheme of FIG. 13A, but further including attachment of pendant tether strands to the antibody. A SpyCatcher modified antibody is contacted to SpyTag-PEG-DBCO molecules at 20° C. for 2 hours, thereby providing a DBCO-modified antibody. The DBCO-modified antibody is then incubated with azide-terminated oligonucleotides for ~12 hours, thereby providing pendant oligonucleotides to the antibody. Subsequently, the antibody is provided to the Phiton attachment process described in FIG. 13A. The resulting product is an antibody with a Phiton fluorescent label and one or more tether oligonucleotides.

### Example 7. Synthesis of Quantum Dot Binding Reagent

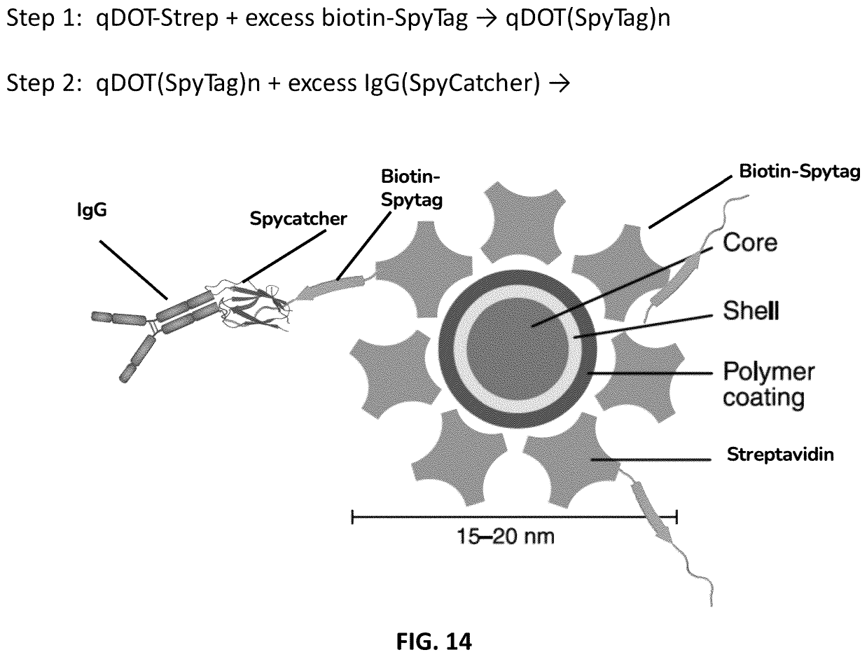

FIG. 14 depicts a binding reagent formed with a quantum dot that is configured to be a linking moiety and detectable label. The quantum dot comprises an inorganic core region surrounded by a shell layer and a polymer coating. The polymer coating is attached to a plurality of monovalent or multivalent streptavidin (SA) molecules. The SA-coated quantum dot is contacted to an excess of biotinylated SpyTag peptides, thereby forming a SpyTag-modified quantum dot. Moieties can be attached to the SpyTag-modified quantum dot in a step-wise or simultaneous reaction. The quantum dot shown is FIG. 14 is contacted with an excess of SpyCatcher-modified antibodies, thereby forming a binding reagent comprising at least one antibody attached to the quantum dot. The quantum dot shown in FIG. 14 could also be contacted with an excess of SpyCatcher-labeled oligonucleotides to attach tether oligonucleotides to the quantum dots. Alternatively, tether oligonucleotides can be attached to the SA-modified quantum dot using biotinylated oligonucleotides. Remaining streptavidin binding sites can be reacted with biotinylated SpyTag molecules before attaching the antibodies.

### Example 8. Binding Reagent with Fluorescently-Labeled Streptavidin

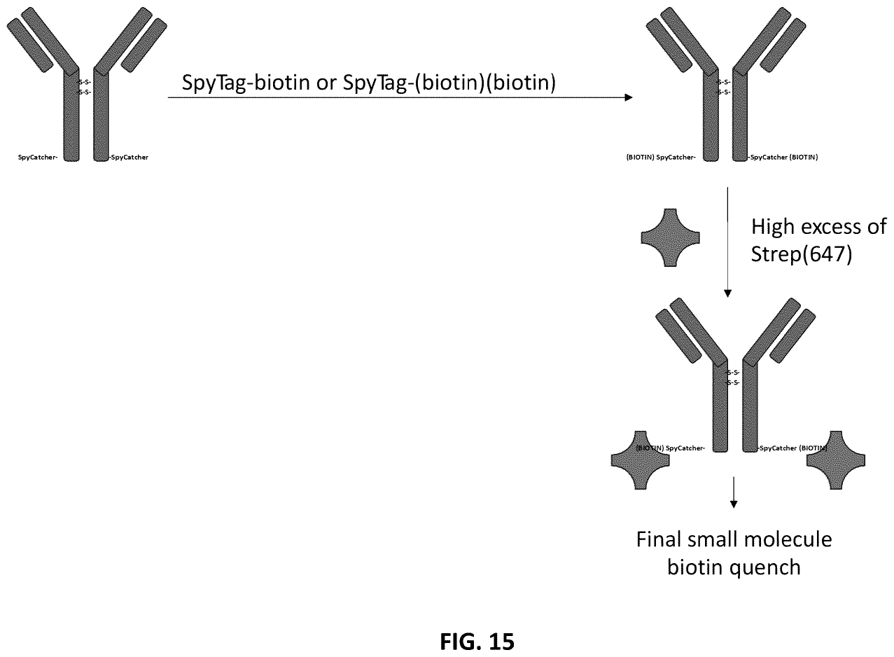

FIG. 15 provides a reaction scheme for forming an antibody binding reagent. A SpyCatcher-modified antibody is reacted with biotinylated SpyTag peptides to form a biotinylated antibody. The biotinylated antibody is then contacted to fluorescently-labeled streptavidin molecules, thereby attaching fluorescent labels to the biotinylated antibody. If available binding sites remain on the attached streptavidin molecules, biotinylated oligonucleotides can be reacted to the streptavidin-modified antibody to provide tether oligonucleotides. Fluorescent streptavidin molecules can be provided by direct attachment of fluorescent molecules to streptavidin, attachment of single biotinylated dyes to streptavidin binding sites, or by attachment of biotinylated clustered dye compositions to streptavidin binding sites.

### Example 9. Clustered Molecule Compositions

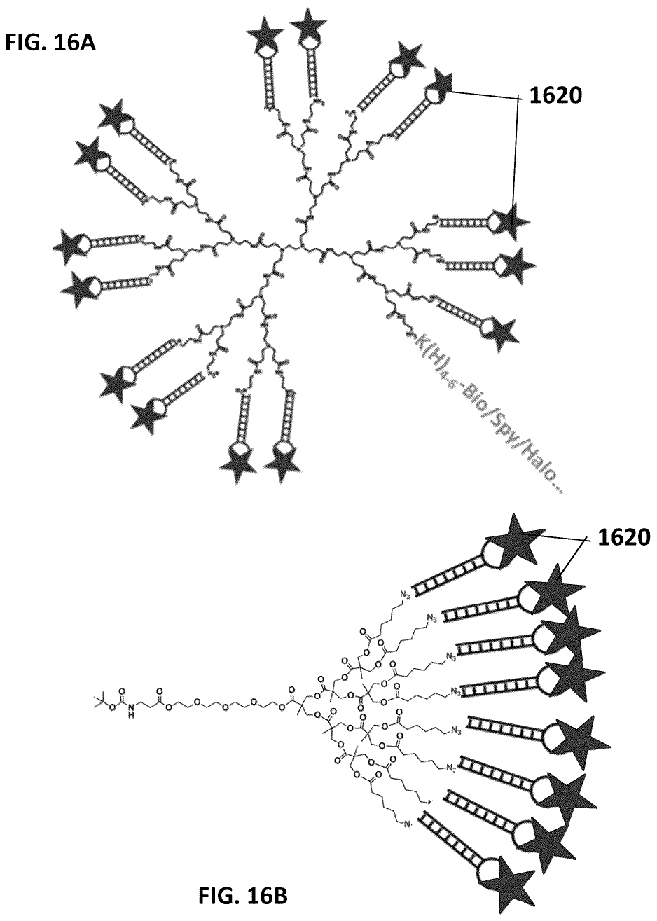

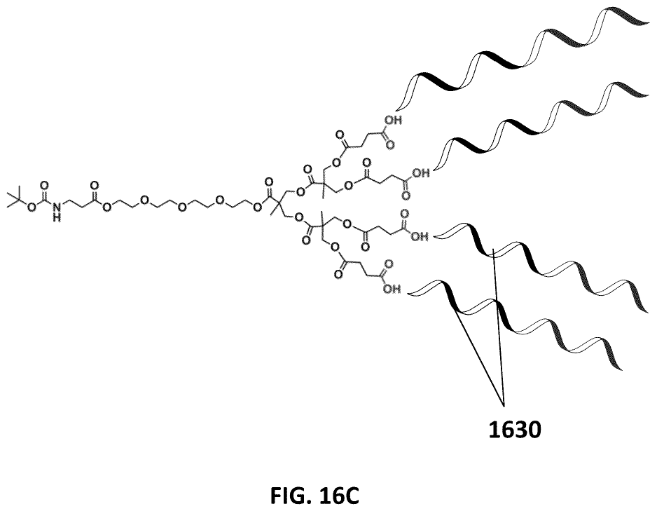

FIGS. 16A-16C depict examples of clustered molecular compositions that may be useful for providing compositions such as clustered detectable labels or clustered tether strands. FIG. 16A depicts a dendrimer-like molecule that has been modified as a clustered fluorescent dye composition. Each terminal primary amine of the dendrimer-like molecule is reacted with a NHS ester-modified oligonucleotide, thereby attaching the oligonucleotides to the dendrimer-like molecule. Each oligonucleotide has a region of self-complementarity, thereby forming a double-stranded nucleic acid with a stem-loop structure. The loop region of each stem-loop contains a modified nucleotide that is attached to a fluorescent dye molecule. The double-stranded nucleic acid regions may provide a degree of repulsion to neighboring nucleic acids, thereby inhibiting dye quenching effects by maintaining sufficient spacing of neighboring dyes. FIG. 16B depicts a similar compositions formed by a dendron-like molecule. The molecules of FIGS. 16A and 16B can further comprise an attachment moiety, such as SpyTag, biotin, or Halo tag, that facilitates attachment to a linking moiety or affinity reagent. FIG. 16C depicts a dendron-like molecule that is attached to a plurality of single-stranded polymers (e.g., oligonucleotides, peptides). A dendron-like molecule with a plurality of terminal primary amines can be reacted with a plurality of NHS ester-modified polymer strands to form the composition of FIG. 16C. The composition may be useful for providing clustered nucleic acid or peptide barcodes, or providing clustered tether strands.

### Example 10. Binding of Tetravalent IgG Molecules to Peptide Targets

IgG antibodies to trimer peptide targets were modified from bivalent antibodies to bivalent antibodies. Each type of IgG antibody was modified to include a second variable region on the heavy chain. The second variable region was separated from the first variable region by an ~30 amino acid linker sequence (either ~15 repeats of the amino acid sequence P-Q or 15 repeats of the amino acid sequence G-Q). Each type of IgG molecule had a total of four paratopes. IgG antibodies modified into a tetravalent form were specific to the following trimer peptide targets: A-T-F, P-I-A, F-T-T, S-T-F, and H-S-P.

EC50 for unmodified and modified versions of the antibodies were measured by enzyme-linked immunosorbent assay (ELISA). For each peptide target, EC50 was measured for the unmodified antibody and the P-Q and G-Q linker versions of the tetravalent antibodies.

Table I provides measured EC50 values for the tested antibodies against the five peptide targets. In Table I, tetravalent antibodies are labeled with “TV” plus the type of linker joining the variable regions, and unmodified antibodies are labeled with “UM.” The tetravalent constructs are generally observed to have lower EC50 values than the unmodified antibodies. Optimal linker type is observed to vary by antibody, although the impact on target binding caused by the linker is far smaller than the impact of introducing the additional paratopes to the antibodies.

| Peptide Target | Antibody Type | EC50 (nM) |
|---|---|---|
| ATF | UM | 21.36 |
| ATF | TV G - Q | 0.64 |
| ATF | TV P - Q | 0.33 |
| PIA | UM | 6.87 |
| PIA | TV G - Q | 0.51 |
| PIA | TV P - Q | 0.096 |
| FTT | UM | 4.45 |
| FTT | TV G - Q | 0.37 |
| FTT | TV P - Q | 0.17 |
| STF | UM | 17.27 |
| STF | TV G - Q | 0.50 |
| STF | TV P - Q | 1.03 |
| HSP | UM | 0.19 |
| HSP | TV G - Q | 0.067 |
| HSP | TV P - Q | 0.082 |

### Example 11. Synthesis of PEGylated Cluster Dyes

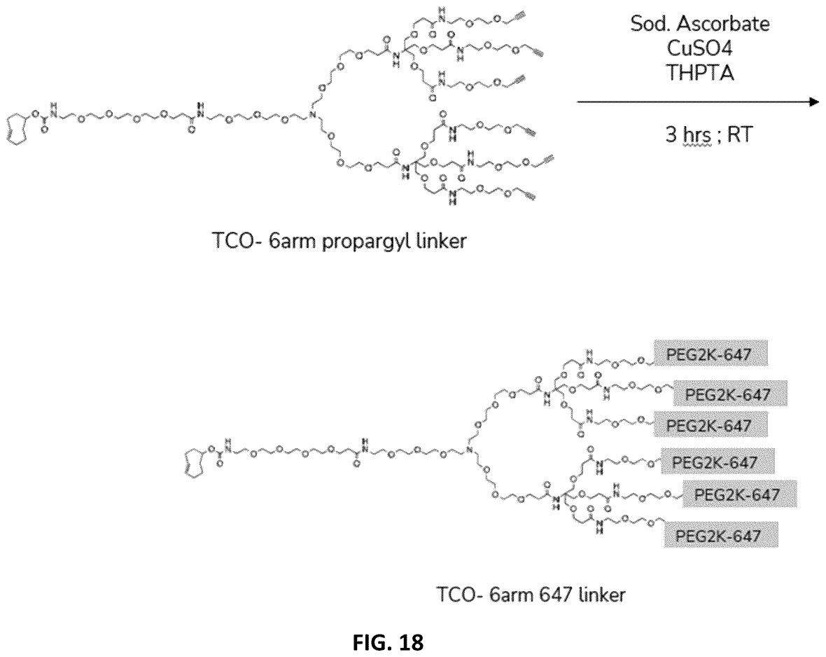

Cluster dyes based on a polyethylene glycol (PEG) scaffold were synthesized. A scheme for the reaction is shown in FIG. 18. A branched PEG chain with 7 arms was provided. On one arm was a trans-cyclooctene (TCO) moiety. The other 6 arms contained terminal propargyl moieties. The PEG scaffold was reacted with Azide-PEG2K-Alexa Fluor 647 dye molecules. The reaction was performed in a 50:50 mixture of water and dimethyl sulfoxide (DMSO) with sodium ascorbate, copper sulfate, and tris(3-hydroxypropyltriazolylmethyl)amine (THPTA) in a 40:25:50 ratio, respectively. The PEGylated dye molecules were provided in five-fold molar excess relative to the PEG scaffold. The reaction was performed at room temperature for 3 hours. After the reaction, click reagents were purified from the crude reaction mixture by wash and buffer exchange with an Amicon 3K molecular weight cut-off (MWCO) column. Thereafter, excess dyes were removed by size exchange chromatography on a Zeba column with a 40K MWCO.

After collection of PEGylated cluster dyes following the SEC purification, the PEGylated cluster dyes were attached to antibodies via a click chemistry reaction between the TCO groups of cluster dyes with terminal methyltetrazine (mTz) moieties attached to the antibodies. The skilled person will readily recognize that the cluster dyes described above can readily be attached to any detectable probe composition set forth herein.

1.-25. (canceled)

26. A method, comprising:
(a) providing an antibody having a binding specificity for an epitope of 2, 3, 4, or 5 amino acids in length; and
(b) attaching a detectable label to the antibody in a site-directed manner, wherein the detectable label comprises a polymer strand and one or more luminophores attached to the polymer strand.

27. The method of claim 26, wherein the antibody comprises a terminal peptide tag.

28. The method of claim 27 wherein the terminal peptide tag comprises a sortase tag.

29. The method of claim 27, wherein attaching the detectable label to the antibody in a site-directed manner comprises enzymatically attaching the detectable label to the terminal peptide tag of the antibody.

30. The method of claim 27, wherein the terminal peptide tag comprises an avi tag.

31. The method of claim 30, wherein attaching the detectable label to the antibody in a site-directed manner comprises: (i) enzymatically attaching a biotin moiety to the avi tag of the terminal peptide tag, and (ii) attaching the detectable label to the biotin moiety, wherein the detectable label further comprises a biotin-binding protein, and wherein the detectable label is attached to the biotin moiety by binding of the biotin-binding protein of the detectable label to the biotin moiety of the terminal peptide tag.

32. The method of claim 27, wherein the terminal peptide tag comprises a polyhistidine tag.

33. The method of claim 32, wherein attaching the detectable label to the antibody in a site-directed manner comprises attaching the detectable label to the polyhistidine tag of the antibody by an imidazole-mediated chelation reaction.

34. The method of claim 26, wherein the detectable label further comprises a photo-catalyzed crosslinking moiety.

35. The method of claim 34, wherein attaching the detectable label to the antibody in a site-directed manner comprises: (i) combining the antibody with the detectable label, (ii) contacting the antibody and the detectable label with a light field, and (iii) in the presence of the light field, attaching the photo-catalyzed crosslinking moiety of the detectable label to the antibody.

36. The method of claim 35, wherein the detectable label further comprises a binding moiety, wherein the binding moiety comprises the photo-catalyzed crosslinking moiety.

37. The method of claim 36, wherein attaching the binding moiety to the antibody comprises attaching the binding moiety to a peptide tag attached to the antibody.

38. A method of forming a detectable probe, comprising:
(a) providing an antibody having a binding specificity for two or more differing proteins of a proteome, wherein the two or more differing proteins of the proteome are not differing isoforms or differing proteoforms of a protein;
(b) attaching a detectable label to the antibody in a site-directed manner, wherein the detectable label comprises a polymer strand and one or more luminophores attached to the polymer strand.

39. A method of characterizing a plurality of analytes, comprising:
(a) providing a solid support comprising the plurality of analytes, wherein the plurality of analytes is immobilized on the solid support, and wherein each analyte of the plurality of analytes is immobilized at a unique address of the solid support such that the analyte is detectable at single-analyte resolution;
(b) contacting a sequence of detectable probes to the solid support, wherein the sequence of detectable probes comprises two or more detectable probes that differ with respect to binding specificity, wherein each detectable probe of the sequence of detectable probes comprises an affinity reagent attached to a detectable label in a site-specific manner, and wherein the detectable label of each detectable probe comprises a polymer strand attached to one or more luminophores;
(c) at each address of the solid support containing an immobilized analyte of the plurality of analytes, detecting a presence or absence of each detectable probe of the sequence of detectable probes; and
(d) based upon the detected presence or absence of binding for each detectable probe of the sequence of detectable probes to each analyte of the plurality of analytes, characterizing each analyte of the plurality of analytes.

40. The method of claim 39, wherein a detectable probe of the sequence of detectable probes has a binding specificity for an epitope of 2, 3, 4, or 5 amino acids in length.

41. The method of claim 39, wherein a detectable probe of the sequence of detectable probes has a binding specificity for two or more differing proteins of a proteome, wherein the two or more differing proteins of the proteome are not differing isoforms or differing proteoforms of a protein.

42. The method of claim 39, wherein a detectable probe of the sequence of detectable probes has a binding specificity for a particular isoform or proteoform of a protein.

43. The method of claim 39, wherein a detectable probe of the sequence of detectable probes has a binding specificity for an epitope comprising a post-translational modification.

44. The method of claim 39, wherein the solid support comprises a plurality of wells, wherein the plurality of analytes is immobilized in the plurality of wells, and wherein each well of the plurality of wells contains only one analyte of the plurality of analytes.

45. A system for analyte characterization, comprising:
(a) a solid support comprising the plurality of analytes, wherein the plurality of analytes is immobilized on the solid support, and wherein each analyte of the plurality of analytes is immobilized at a unique address of the solid support such that the analyte is detectable at single-analyte resolution;
(b) one or more vessels, each vessel of the one or more vessels comprising a detectable probe composition, wherein each detectable probe of the detectable probe composition comprises an affinity reagent attached to a detectable label in a site-specific manner, and wherein the detectable label of each detectable probe comprises a polymer strand attached to one or more luminophores;
(c) a fluidics system, wherein the fluidics system provides fluidic communication between the solid support and the one or more vessels;
(d) an optical detection device; and
(e) a processor in communication with the optical detection device, wherein the processor is configured to receive signal information from the optical detection device and determine the presence or absence of a detectable probe of the detectable probe composition at each unique address of the solid support.
